(19) United States
(12) Patent Application Publication
(10) Pub. No.: US 2022/0387249 A1
(43) Pub. Date: Dec. 8, 2022

(54) THERAPEUTIC COMPRESSION APPARATUS, SYSTEM AND METHODS OF USE

(71) Applicant: Sun Scientific, Inc., Dobbs Ferry, NY (US)

(72) Inventors: Sundaram Ravikumar, Dobbs Ferry, NY (US); Vikram Ravikumar, Dobbs Ferry, NY (US)

(73) Assignee: Sun Scientific, Inc., Dobbs Ferry, NY (US)

(21) Appl. No.: 17/770,200

(22) PCT Filed: Oct. 23, 2020
(86) PCT No.: PCT/US20/57224

§ 371 Date: Apr. 19, 2022

Related U.S. Application Data
(63) Continuation of application No. 62/924,871, filed on Oct. 23, 2019.

(51) Int. Cl. A61H 9/00; A61F 13/08

(52) U.S. Cl. CPC A61H 9/0092; A61F 13/08; A61H 2201/1642; A61H 2201/165; A61H 2201/1638; A61H 2201/163

(57) ABSTRACT

A therapeutic compression system for providing gradient compression to a limb or other body part, including at least one therapeutic compression apparatus having at least one elastic sock or sleeve integral and connected to at least one wrap having at least one bladder and a universal inflation port to connect to an inflation means to the at least one bladder. The connecting means connects the at least one elastic sock or sleeve in one embodiment by buttons inserted and connected into apertures on the at least one wrap. The inflation port has an open and closed mechanism so that when closed it maintains the level of inflation in the at least one bladder. The inflation means provides either constant or varying pressure within the bladder and a check valve is included in the inflation port and/or inflation means. The inflation means may be connected to a stabilization means configured to be attached to a body part of the user or a belt or bag or other means around for example, the shoulder, leg, waist or arm of the body. The inventive therapeutic compression apparatus and system is stabilized and secured on the user when the user moves the target body part.

### RELATED APPLICATIONS

The present application claim priority from PCT/US20/057224 filed Oct. 23, 2020 and provisional application Ser. No. 62/924,871 filed Oct. 23, 2019 and both entitled “Therapeutic Compression System and Methods of Use”, the entire contents of which are hereby incorporated herein by reference for all purposes.

### BACKGROUND OF THE INVENTION

### I. Field of the Invention

The subject invention is directed generally to a device for applying compression to a limb, and more particularly, to a therapeutic apparatus for applying compression to an extremity of an individual such as by example a leg or arm in conjunction with the treatment of conditions such as deep vein thrombosis (DVT), chronic venous insufficiency (CVI) and lymphedema, and more specifically to a therapeutic compression apparatus for including a connected sleeve or sock. The apparatus includes a limb covering, such as a knee-high sock or arm sleeve, integral with a bladder and an inflation means for the bladder with the apparatus capable of applying compression to a limb such as the full leg, thigh, calf, knee, foot, arm, shoulder or other limb of a patient.

### II. Background of the Related Art

Normally, a healthy leg muscle, for example, squeezes the deep veins of the legs and feet to help move blood back to the heart. One-way valves in the deep leg veins keep blood flowing back towards the heart. However, prolonged periods of standing or sitting can cause the walls of the deep leg veins to stretch. Over time, in susceptible individuals, this can weaken the vein walls and damage the valves, causing blood to pool in the veins and increase venous blood pressure. This may result in a condition known as chronic venous insufficiency (CVI).

Surgery may also increase the possibility of CVI and/or lymphedema including hip surgery, knee surgery, shoulder surgery, elbow surgery and breast cancer surgery among other types of surgery. In the upper body the extremity of the arm and hand can be subject to CVI and/or lymphedema due to the surgery itself and then recovery periods when the arm or hand has to remain still and typically elevated. If the hand, arm, elbow, shoulder or upper body are not elevated the blood will typically pool at the position that is lower in gravity and not flow back up to the shoulder and heart area. All of the above surgeries, recoveries, treatments and conditions can lead to a new condition or exasperate an existing condition of CVI and/or lymphedema.

Treatment of CVI typically involves the use of compression sleeves, stockings or medical hosiery to decrease chronic swelling. Compression sleeves or stockings are elastic material that squeeze the veins to improve venous circulation and prevent excess blood from flowing backward. Compression sleeves or stockings can also help to heal skin sores or stasis ulcers that often present in conjunction with CVI. It is also common to employ compression bandages to apply pressure to the leg or arm (as well as other limbs of the body). In this regard, a bandage is applied with constant tension so as to produce graduated compression with the highest pressure at the ankle, hand, or other are of the body which is lower in placement from the heart and return vein. However, the technique is difficult and is often done by highly skilled caregivers.

Highly effective mechanical compression devices have also been developed for treating CVI, which are disclosed, for example, in U.S. Pat. Nos. 7,276,037 and 7,559,908, the disclosures of which are incorporated by reference herein in their entireties. These devices include a flexible wrap that carries a manually inflatable air bladder and is adapted to be securely positioned around the leg of an individual to apply localized pressure to a treatment site. The device also includes a fluid-filled wound dressing that can be applied directly to the skin for applying localized pressure and even a medicament to a venous nicer when it is enveloped by the flexible wrap. While this device is effective for applying localized compression to the leg, it is not configured to apply localized compression to the foot to prevent swelling and further improve venous circulation.

Lymphedema, also known as lymphatic obstruction, is another condition of localized fluid retention and tissue swelling, and is caused by a compromised lymphatic system. Treatment for lymphedema varies depending on the severity of the edema and the degree of fibrosis of the affected limb. The most common treatments for lymphedema are manual compression lymphatic massage, compression garments or bandaging. Elastic compression garments are typically worn by persons with lymphedema on the affected limb following complete decongestive therapy to maintain edema reduction.

Compression bandaging, also called wrapping, involves the application of several layers of padding and short-stretch bandages to the involved areas. Short-stretch bandages are preferred over long-stretch bandages (such as those normally used to treat sprains), as the long-stretch bandages cannot produce the proper therapeutic tension necessary to safely reduce lymphedema and may in fact end up producing a tourniquet effect. During activity, whether exercise or daily activities, the short-stretch bandages enhance the pumping action of the lymph vessels by providing increased resistance for them to push against. This encourages lymphatic flow and helps to soften fluid-swollen areas.

Known methods for CVI and lymphedema treatment, like compression bandaging, have several disadvantages. The bandaging is time consuming and the effectiveness is limited to the skill of the provider. In some instances, bandages can be applied too tightly or too loosely and may slip from their intended position, decreasing their effectiveness. When this occurs, bandages must be taken off and reapplied, further increasing the time of application and decreasing the consistency of application of the therapy. Further, when the compression bandaging is on the thigh area of the leg, the bandaging slips down the leg when the user walks or moves around even in bed, possibly due to the anatomical shape of the thigh tapering towards the knee area. If the compression bandage is on the lower leg gravity and again the calf tapering towards the ankle results in the bandage slipping downward which is uncomfortable and more importantly lessen the treatment due to loosened tightness and less compression. If the compression bandage is on the arm once again gravity and the bicep upper arm tapering towards the elbow and then the lower arm towards the wrist results in the bandage slipping downward which is uncomfortable and more importantly lessen the treatment due to loosened tightness and less compression.

The effectiveness of many of the current compression therapies is limited by the application of current products. Because current compression therapy is done either with manual wraps or electromechanical systems, they require either a skilled medical processional to apply and/or the need for the patient to be stationary for extended periods of time. It is exceeding difficult, if not impossible, for the user to apply the compression therapy by himself or herself, and even more difficult if the user has reduced mobility of the hand(s), arm(s), back and leg(s). Further, the sleeves, stockings and/or bandages will slip down the limb towards either the knee, ankle, elbow or wrist once the user sits up and/or walks or moves around. The same problem would be seen if the sleeve, stocking and/or bandage was originally placed on the torso, waist, neck, hips or any other limb when due to gravity and the user's movement the sleeve, stocking and/or bandage will slip downward and lessen the actual compression on the treatment site.

CVI and lymphedema may also result in DVT, which is an affliction that causes blood clots particularly in the lower extremities of the legs but also affects the arms and torso. When a patient is not ambulatory, the patient faces an elevated risk factor of creating a blood clot. The individual may experience blood clots in the hospital but also at home, on a plane or car ride and any other situation where the individual is not moving for a period of time. These blood clots, which often accumulate or reside in the patient's calf, thigh, arm or torso (as well as many other body parts) are not, in and of themselves, overly dangerous. However when the blood clot breaks loose, they create a pulmonary embolism which can get lodged in the patient's heart, brain or lungs where it can cause significant damage or death. It is estimated that in each year 2 to 2.5 million Americans are afflicted by DVT causing 600,000 patients to seek medical care with 300,000 patients succumbing to the effects of the pulmonary embolism. Thus, a system and apparatus and method of use to treat DVT is needed.

Many of the current treatment options for CVI and lymphedema cause venous ulcers including the use of current known devices, apparatus, bandages, stocking, sleeves, hosiery and the like. A venous ulcer is damage and loss of skin typically above the ankle that is the result of a problem with the veins in the leg. Venous ulcers typically develop on either side of the lower leg, above the ankle and below the calf. They are difficult to heal and often recur. They also develop on the thigh, on the inner portion of the leg thigh or outer thigh area as well as near the groin area on the leg. Venous ulcers can also develop on the wrist, elbow or arm area of a patient.

The veins of the leg are divided into the superficial and deep systems according to their position relative to the fascia. The deep veins, which come together to form the popliteal and femoral veins lie within the fascia and are responsible for the venous return from the leg muscles. Dilated valveless sinusoids also lie within the fascia (more particularly in the soleus and gastrocnemius muscles). The sinusoids fill with blood when the leg is at rest. The long saphenous vein which runs along the medial side of the leg from foot to groin and the short saphenous vein which runs at the back of the calf from foot to knee are the major vessels of the superficial venous system. These vessels lie outside the fascia and are responsible for the venous return from the skin and subcutaneous fat. Communicating veins, sometimes called perforators because they perforate the deep fascia, join the two systems. The perforators, like the other veins in the leg, contain valves that permit the flow of blood in one direction only, from the outer or superficial system inwards to the deep veins.

The venous pressure at the ankle of a subject who is lying supine is around 10 mm Hg, but on standing this will rise considerably due to an increase in hydrostatic pressure (equivalent to the weight of a vertical column of blood stretching from the point of measurement to the right auricle of the heart). During walking, as the foot is dorsally flexed, the contraction of the calf muscle compresses the deep veins and soleal sinuses thereby emptying them of blood. As the foot is plantarly flexed, the pressure in the veins falls, the proximal valves close, and the veins are refilled by blood passing through the perforators from the superficial system. During this cycle, in a normal leg, the distal valves of the deep veins and the valves of the perforators will ensure that the expelled blood can go in only one direction—upwards, back to the heart.

Blockage or damage to the venous system will cause disruption to normal blood flow, which may manifest itself in a number of different ways according to the site and extent of the damage. If the valves in the superficial system are affected, venous return will be impaired and blood may accumulate in the veins causing them to become distended, leading to the formation of varicosities (varicose veins). Such varicosities may be located in the thigh, knee, calf, ankle or foot area of the user's leg. The same can occur in the arm and torso or chest as well as other limbs and extremities. If the function of the perforator valves is impaired, the action of the calf muscle pump will tend to cause blood to flow in the reverse direction into the superficial system increasing the possibility of damage to the superficial vessels.

Following a deep vein thrombosis that results in complete or partial obstruction of a deep vein, the unrelieved pressure produced by the calf muscle pump on the perforator valves may cause these to become incompetent. If this occurs, there will be a large rise in the pressure in the superficial system, which may force proteins and red cells out of the capillaries and into the surrounding tissue. Here, the red cells break down releasing a red pigment that causes staining of the skin, an early indicator of possible ulcer formation.

Venous leg ulcers are generally shallow and red in color. The skin surrounding the ulcer is frequently discolored due to the staining described previously. Incompetent perforating vein valves can also cause malleolar venules to become dilated and appear as fine red threads around the ankle as well as the thigh, knee, calf or foot are of the leg or any area of the leg. This condition, called ankle flair, is also diagnostic of a venous ulcer.

Arteries transport oxygen replenished blood from the heart to the rest of the body. Veins return oxygen depleted blood back to the heart. When the veins in the lower or upper extremities of the body have difficulty transporting blood back to the heart, a condition develops called chronic venous insufficiency (CVI), also known as chronic venous disease (CVD). CVI most commonly occurs as the result of a blood clot in the veins which can be in the arm or hand but more typically in the deep veins of the legs, leading to a disease known as deep vein thrombosis (DVT). CVI also results from pelvic tumors and vascular malformations, and sometimes occurs for unknown reasons. When a person is standing or sitting, blood in the veins of the legs and arms flows in an upward direction. When the person walks, the calf muscles and muscles in the feet contract to squeeze the veins and push the blood upward. To keep the blood flowing upward and prevent it from flowing downward, the veins contain one-way valves. CVI occurs when these valves, whether in the arms or legs, become damaged and allow the blood to leak back downward in the opposite direction. Such valve damage may occur as the result of aging, extended sitting or standing, or a combination of aging and reduced mobility. Such causes can include surgery in a young otherwise healthy person as well. When the veins and valves become weakened and the blood does not properly flow up to the heart, blood pressure in the veins of the upper and lower extremities can stay elevated for long periods of time, leading to CVI. This condition is more common in older individuals, and if not properly treated, can lead to burst capillaries, local tissue inflammation, internal tissue damage, varicose veins, ulcers, and open sores on the skin's surface. However, surgical procedures or other medical treatments can also contribute to CVI and DVT, or remaining stationary in a pressurized airplane or even staying seated in an extended car or train ride. There is a long-felt need for a compression apparatus that can reduce the symptoms of lymphedema, swelling, venous ulcers, CVI, DVT or other conditions which is easier to place on the target limb or body part by the user alone without a skilled medical provider, especially if the user has reduced mobility including without limitation trouble using his or her hands, arms, legs, back, and any other body part.

Further there is a long-felt need for a compression apparatus, device or garment that the user can place on a target body area and then affix or close the garment around the target body area, such as with straps, hook and loop fasteners, or VELCRO™. For example, there are known conventional hook and loop fastener (such as VELCRO™ straps) compression garments typically without a compression sock or stocking but the user has trouble placing the garment onto the target body area and then closing the fasteners, whether the garment has compression or not. In use, the garment may have one or more set of hook and loop fasteners and the user, with possible reduce mobility hands or legs or back or reduced strength, has to attempt to locate the garment on the target body area and then close the hook and loop fasteners (such as VELCRO™ straps). As the user tries unsuccessfully to close one hook and loop fastener (such as VELCRO™ strap), the other hook and loop fasteners (such as VELCRO™ straps) are moving off the target body location and the entire garment moves around off the target body part. Again, this is hard to accomplish sufficiently by a skilled care-giver but extremely difficult if not impossible if the user has reduced strength in his or her hands, or legs and can't bend over to place the garment in the open position onto the foot or leg or thigh and locate it onto the target body part and then simultaneously close the garment by closing the hook and loop fasteners (such as VELCRO™). Straps) or other closing means. The user has an even more difficult time placing the garment onto one arm, locating it at the target body part and then closing it only using the other hand. Thus a need exists for a compression garment which is easier for the user to place on the target body location and then close or apply compression.

CVI can diminish the capacity of the venous system and increase the workload of the lymphatic system in the affected area. The lymphatic system must then transport larger volumes of water and protein to reduce the fluid load in the affected tissues of the legs or arms or other body parts, a situation which is especially difficult for patients with lipedema, varicose veins, and other lower or upper extremity pathology.

One non-surgical option often used to help prevent or treat the leg and arm extremity pathologies discussed above is the use of compression stockings. Compression stockings are not limited to foot stockings but include arm stockings or sleeves or any other type of compression sleeve or material for placement on a body part including without limitation a foot, ankle, calf, leg, knee, thigh, hip, pelvis, torso, chest, shoulder, arm, elbow, wrist, hand, back, neck or other body parts. Compression stocking or sleeves on the leg help prevent leg fatigue, ankle and foot swelling, spider veins, and varicose veins. Compression stocking or sleeves on the arm help prevent arm fatigue, elbow and wrist and hand swelling, spider veins, and varicose veins. They improve circulation in the body part, especially when used in conjunction with frequent exercise and body part elevation. Compression stockings or sleeves maintain pressure on the body part while allowing for normal ambulation. Increasing pressure in the tissues beneath the skin reduces excess leakage of fluid from the capillaries and increases absorption of tissue fluid by the capillaries and lymphatic vessels. In addition, the increased pressure decreases the size of the veins, which causes the blood to flow faster and help prevent it from pooling. Compression stockings or sleeves can also be used by individuals after exercise or working out for recovery purposes. They can be used to treat the symptoms of lymphedema, CVI, DVT or even conditions associated with swelling of the arm during dialysis.

Compression stocking or sleeve tightness typically varies between 15-50 mm HG. The tightness of a given stocking or sleeve depends on its particular configuration and class. For example, one type of stockings having a compression pressure of 15-20 mm HG are considered light compression stockings. Class I stockings are 20-30 mm Hg, class II stockings are 30-40 mm Hg, and class III stockings are 40-50 mm Hg. The same classes are applicable to compression sleeves or other compression embodiments. While such compression stockings or sleeves are a commonly utilized non-invasive treatment of leg pathology, the issues they present are numerous. Wearing a tightly fitting stocking or sleeve can be tedious or time consuming to put on, and may require help from another person if the wearer is injured, elderly, or has some form of disability or reduced mobility in the arms, hand, legs, back and other body parts. For example, any tight fitting stocking or sleeve to be worn on the thigh area has a harder time being put on as the user has to pull the tight fitting stocking over the foot, ankle, calf, knee and finally up to the thigh area, which may be difficult for the user if his or her hands have reduced mobility or reduced strength. Again, due to the shape of the thigh, these tight fitting stocking or sleeve will slip down the thigh at some point due to the user walking or moving about. The same is true for a tight fitting stocking or sleeve on the calf or lower leg as the user has to pull the compression stocking or sleeve over the foot and ankle and then up the calf and then further repeat this action if the compression stocking or sleeve slips down from the knee to the ankle when the user is walking or moving about. This is particularly a problem for the user if injured and cannot pull the compression stocking or sleeve over the extremity or if the user has reduced capacity of the hands to pull onto the leg or arm, or reduced capacity to lean over to place the compression stocking or sleeve on the foot and then pull up the leg. Notably, if the user is placing the tight compression stocking or sleeve on one arm it is extremely difficult if not impossible to use one hand to place the compression stocking or sleeve on the other hand and then pull up the arm. There is a long-felt need for a compression apparatus which can be placed easier on a body part by the user himself or herself and which can be placed at the location for compression to be applied to the target body are such as a foot, ankle, leg, calf, knee, thigh, hip, waist, back, torso, shoulder, arm, elbow, upper arm, forearm. wrist, hand, neck or any other body part.

In addition, the pressure applied by the stocking or sleeve generally stays relatively constant during use without any option of increasing or decreasing the tightness level. As compression stockings or sleeves are repeatedly worn, they lose elasticity and thus tightness over time. Once such prescribed elasticity and tightness is lost, the stocking or sleeve is of little or no value, and needs to be replaced on account of its looseness, which requires buying a new one or pair to obtain the desired pressure. Further, given the shape of the body part the compression stocking or sleeve may have to be very tight. For example, a compression stocking or sleeve on the thigh typically has a very tight top or upper portion in order to prevent slippage down the thigh, which can in turn lead to complications of its own due to the excessive tightness. Given the shape of the lower leg, the top or upper portion closer to the knee is one circumference, with the calf being a larger circumference and then the ankle is the smallest circumference resulting in three different sizes on one stocking or sleeve. Similarly, the shape of the upper arm and lower arm are also tapered in a downward direction with the uppermost arm near the shoulder being a larger circumference compared to nearer the elbow and the same for the lower arm circumference being greater nearer the elbow compared to the wrist. Again, the compression stocking or sleeve will typically have one compression level so that it will be tighter in the area have a larger circumference compared to the area having a smaller circumference. This itself can lead to problems and complications of swelling and affect the artery and vein blood flowing.

Medical hosiery represents a useful and convenient method of applying compression to normal shaped legs in order to prevent the development or recurrence of leg ulcers. However, these stockings or sleeves are of limited value in the treatment of active ulceration, being difficult to apply over dressings. In such situations compression bandages currently represent the treatment of choice. Compression bandages apply a pressure to the limb that is directly proportional to bandage tension but inversely proportional to the radius of curvature of the limb to which it is applied, whether the limb is a leg or arm or other body part. This means, therefore, that a bandage applied with constant tension to a limb of normal proportions will automatically produce graduated compression with the highest pressure at the knee or a leg or elbow of an arm. This pressure will gradually reduce up the thigh or arm as the circumference increases. Likewise, given the larger circumference of the calf the pressure will reduce along the calf versus the knee or ankle and likewise the pressure will reduce along the arm versus the wrist or elbow. As can be readily appreciated, it is cumbersome and difficult to apply uniform tension to the compression bandage as it is applied to the treated limb, and thus this is accomplished only by highly skilled caregivers. Moreover, once secured to the treated limb, care and attention must be given to ensure that the bandage does not slip or become displaced as this will lead to multiple layers forming, which in turn may lead to localized areas of high pressure, which can place the patient in direct risk of skin necrosis.

Other known compression garments may include wraps that apply pressure or compression to the target body part by closing up straps or pulling a strap tighter at the target body part or location on the limb, or closing hook and loop fasteners such as VELCRO™ again around the target body part. If the user is attempting to locate and place on the target body area a garment or wrap having straps without the assistant of a skilled care-giver or medical provider, the user has to simultaneously place the garment on a location on the target body area while at the same time closing the straps and possibly tightening the straps, all concurrently as the garment keeps falling down on the foot, leg, knee, thigh, hip, torso, arm, elbow, chest, shoulder, neck, etc. This frustrating actions are exacerbated if the user has reduced strength in his or her hands or can't bend down to place the garment on a lower extremity or has to place the garment on one hand or arm and can only use the other hand to do so. For example, a patient has to place a garment or wrap with straps onto a lower leg and the user has to locate the garment and the straps (which create the compression element when closed and tightened) onto the lower leg at the proper location on the target body part while at the same then closing the straps, hook and loop fasteners, buckles, snaps, VELCRO™ and then again tighten to create sufficient compression on the target body area. Now assume the user has reduced strength in his or her hands and cannot bend down fully due to back issues or has an injured body part, then the ability to successfully place the garment in a location on the target body part and close to create sufficient compression is near impossible. Thus a need exists for a compression apparatus, garment or system where the user can place the garment or apparatus at a location on the target body part which stays on the target body part or is stabilized on it and configured so the user can simultaneously close the straps or fasteners and create sufficient compression to the target body part.

Mechanical compression treatments have also been proposed. An exemplary compression device is described in U.S. Pat. No. 5,031,604 to Dye. As generally described at col. 2, lines 33 et seq., an arrangement of chambers are provided that circumscribe the leg. An active pneumatic control system controls the pressure in the chambers to squeeze the leg near the ankle and then squeeze sequentially upward toward the knee in order to move blood from the extremity toward the heart. As noted in col. 4, lines 20-59 of U.S. Pat. No. 6,488,643 to Tumey et al., the mechanically produced compression levels may produce ischaemic (i.e., localized tissue anemia) not noted at similar compression levels obtained through bandaging. It may also produce cuffing (i.e., a reduction in leg pulsatile blood flow). The pneumatic control system is also bulky and heavy, which severely limits the mobility of the patient during treatment. Moreover, the pneumatic control system fails to provide a mechanism to ensure that excessive pressure, which can cause necrosis, is not applied to the treated limb. These limitations have resulted in most mechanical compression devices being contraindicated for patients exhibiting DVT. Consequently, those skilled in the art have to date avoided such mechanical compression devices for the treatment of venous ulcers or edema of the extremities, upper and lower extremities.

Co-owned U.S. Publication No. 2004/0193084, which is hereby incorporated by reference herein in its entirety, discloses a device for applying pressure to the human leg for use in conjunction with treatment of varicose veins. The device includes a flexible member and at least one air bladder chamber integral thereto that are adapted to securely wrap around the human leg. A tube in fluid communication with the air bladder chamber(s) extends to an air pumping mechanism that operates to inflate the air bladder chamber(s) to a pressurized state. The flexible member preferably includes an opening at the knee joint level to enable a patella to protrude therethrough. In addition, the flexible member preferably extends below knee joint level and is adapted to securely wrap around a lower portion of a leg to provide stability to the leg. Preferably, the air bladder chamber of the device is substantially longer in a first dimension than in a second dimension orthogonal thereto such that the air bladder chamber can be positioned to cover a portion of the human leg that is relatively long in the vertical dimension and narrow in the horizontal dimension. This apparatus does not include an attachable or integral compression stocking or sleeve and is sometimes harder for the user to place onto his or her own leg without assistance depending on the user's own mobility or reduced use of his or her own hand, back or leg.

Co-owned U.S. Pat. No. 7,276,037, which is hereby incorporated by reference herein in its entirety, discloses an apparatus for applying compression therapy to an extremity of the human body, such as a portion of the human leg. The device includes a flexible member and an air bladder chamber. The flexible member is adapted to wrap around the extremity to secure the air bladder chamber to the extremity. An air pumping mechanism is operated to inflate the air bladder chamber to a pressurized state. One or more fluid-filled pressurized members are provided, each separate and distinct from the flexible member and the air bladder chamber and thus readily moveable relative to the flexible member and the air bladder chamber. The pressurized member(s) is operably disposed between the extremity and the flexible member whereby it applies increased localized pressure to the extremity during use. Preferably, the air bladder chamber is substantially longer in a first dimension than in a second dimension orthogonal thereto such that it can extend longitudinally along the extremity to cover a relatively long and narrow portion of the extremity. The position of the air chamber can be readily adapted to apply local pressure to desired body parts (such as a certain venous channel). The pressurized member(s) can be positioned during use such that it covers a venous ulcer (or other treatment sites) and applies increased localized pressure to the treatment site in order to promote healing. This apparatus does not include an attachable or integral compression stocking or sleeve and is sometimes harder for the user to place onto his or her own leg without assistance depending on the user's own mobility or reduced use of his or her own hand, back or leg.

Surgical treatments for leg vascular issues include Sclerotherapy which is a medical procedure used to eliminate varicose veins and spider veins. Sclerotherapy typically involves an injection of a solution (generally a salt solution) directly into the vein. The solution irritates the lining of the blood vessel, causing it to collapse and stick together and the blood to clot. Post-operative procedures may require compression bandaging or stockings or sleeves, all of which have the problems described above in regard to pulling the stocking or sleeve over the foot or wrist and placement onto the appropriate limb such as a leg or arm, slippage down the thigh to the knee or down the calf to the ankle, or slippage down the arm to the elbow or wrist area, as well as cost and need for a skilled level of knowledge in order to properly bandage the sclerotherapy treatment area.

Surgical treatments which may affect the arm area include without limitation breast cancer surgery (removing nodes or a partial or full mastectomy), radiation, shoulder replacement, rotator cuff surgery, SLAP (Superior Labrum Anterior and Posterior) Lesion repair surgery, Biceps Tenodesis shoulder surgery, and any type of shoulder, elbow, wrist or hand surgery. Any of these surgeries may affect vein flow and cause lymphedema, CVI or DVT.

In regard to lymphedema following surgery it can occur subsequent to any of the following and occur shortly after surgery or weeks or months or even years later. Examples of such surgical procedures causing lymphedema include without limitation: simple mastectomy in combination with axillary (arm pit) lymph node removal; lumpectomy in combination with axillary lymph node removal; modified radical mastectomy in combination with axillary lymph node removal; combined cancer surgery and radiation therapy to a lymph node region (such as the neck, armpit, groin, pelvis, or abdomen); or radiation therapy to a lymph node region. Lymphedema may be the result of an abnormal accumulation of lymph in the arm, shoulder, breast, or thoracic area that usually develops within three years of a breast cancer diagnosis but can occur much later.

Lymphedema in the arm may be a chronic condition where the lymph fluid in an extremity of the arm doesn't move as it should because of blockage of the lymph vessels, or a reduction of said vessels. For some individuals, it is a condition that lasts for a period of time after surgery while others have it for the rest of their lives. It is commonly a side effect of removal of lymph nodes, removed either as a precautionary measure or in order to keep cancer from spreading into other parts of the body if cancer was detected in the lymph nodes during a biopsy. While removing the lymph nodes can halt the cancer's movement, it can also reduce the ability of the arm to naturally move the lymph fluid back into the larger lymph vessels. Lymphedema's most noticeable symptom is swelling of the hand, wrist and arm but can also produce tingling and pain in the arm.

It has been reported that arm lymphedema develops in 10%-35% of patients who undergo axillary dissection and/or nodal radiation therapy for breast cancer. Lymphedema that occurs in the first 18 months after surgery or radiation is described as acute lymphedema, and can be managed with conservative measures such as elevation of the arm and mild compression. Chronic lymphedema, the more serious form, has a progressive and generally irreversible course. Risk factors associated with the development of lymphedema include greater extent of axillary surgery; more positive axillary nodes; a postoperative axillary hematoma, seroma, or infection; and use of nodal radiation. The most common method of lymphedema measurement is the circumference 10 cm above and below the olecranon process, although most clinicians do not take measurements in the preoperative setting for comparison. Treatment strategies include elevation, complete decongestive physiotherapy, pneumatic pumps, and, after failure of all other methods, surgery. Lymphangiosarcoma is a rare and late complication of longstanding extremity lymphedema. The advent of sentinel lymph node biopsy as an alternative to axillary dissection should decrease the rate of lymphedema but even this biopsy procedure can lead to lymphedema in patients. The increasing number of breast cancer survivors and the high prevalence of the disease will continue to make lymphedema a significant consequence of breast cancer treatment.

Following a surgery, for cancer or otherwise, the patient typically is treated with compression in the form of bandaging. As with leg compression bandages, the tension and compression required to deal with the lymphedema, CVI or DVT requires skill. Further, the patient has only one hand at that time to use for bandaging on his or her own, versus having two hands to bandage a lower leg or other body part as discussed above. Even if trained in how to bandage correctly with sufficient pressure and compression, most patients cannot bandage the injured or surgical site arm with only one other hand. Thus at home compression bandaging of the arm by the patient himself or herself is not likely effective.

The patient will have similar problems with certain compression garments, wraps and devices if only able to use one hand in order to pull up the compression garment, wrap, stocking, sleeve or device and then close it or set it on the injured arm. The reduced mobility of the user of only being able to use one hand in this instance means the user has to rely on another individual to assist, unless provided with a compression garment or device that can be pulled, set and closed on the injured arm and having sufficient compression or pressure for the lymphedema, CVI or DVT treatment. Thus, a long felt need exists for a compression garment, stocking, sleeve, apparatus, device or system that can be utilized with only one hand of the user while sufficiently pulling the compression garment, stocking, sleeve, apparatus, device or system up on the injured other arm past the hand, wrist, and elbow then securing it in place and applying the required compression or pressure to treat the arm's lymphedema, CVI or DVT.

Further, a need exists for an arm compression garment, stocking, sleeve, apparatus, device or system that maintains stability or stays securely on the injured arm without moving in a downward direction due to gravity and/or the tapering nature of the upper arm or lower arm. Certain apparatus are known that include straps across the torso or chest of the user but they are bulky and uncomfortable to wear for periods of time.

In regard to thigh compression garments or lower leg compression garments, such known compression garments for the user's thigh have a tendency to slip down the user's leg so that the garment either bunches at the knee or falls down the thigh towards the knee and rests above the knee rather than midway on the thigh or has a tendency to slip down the user's calf so that the garment bunches at the ankle. The location of the thigh or lower leg compression garment should remain in place and any movement due to gravity as the user walks can affect the efficacy of the treatment for CVI, DVT and/or lymphedema, as well as other treatments. The thigh region may be the area of a user's leg between the hip and knee and the lower leg region the area of a user's leg between the knee and ankle or foot. The basic anatomy of the leg may cause the slippage in that part to the shape of leg anatomy conical in nature and facilitates slippage in a downward direction of the leg. The same is true for the user in regard to an arm compression garment whereby the compression garment can slip in a downward direction from the shoulder area to the elbow area and from the elbow area to the wrist or hand area.

Users have attempted to reduce slippage of a thigh wrap downward toward the knee or reduce slippage of an arm wrap downward by tightening the wrap as much as possible, but this can result in cutting off circulation and other complications. Some users have added adhesive tape to adhere the thigh wrap or lower leg wrap or arm wrap (whether it is a bandage or compression garment) to the user's skin, but this also can result in complications. Thus, a need exists for a thigh compression garment with reduced slippage down to the knee and a lower leg compression garment with reduced slippage down to the ankle or foot and an arm compression garment with reduced slippage down to the elbow or wrist or hand. A need exists for a compression garment with reduced slippage due to gravity. The same is true for a lower leg compression garment wherein the garment will have reduced slippage from the knee towards the ankle and for an arm compression garment with reduced slippage from the shoulder towards the elbow or from the elbow to the wrist or hand.

All current known treatment apparatus, devices, bandages, stockings and hosiery have the problems of stability (no slippage), maintaining sufficient effective pressure without overpressure complications, maintaining compression and the like. Further all known apparatus, devices, bandages, stockings and hosiery, though especially the current treatment apparatus and devices, are only capable of connecting to one source of compression or inflation means and no universal inflation port of connector is known -wherein a patient could vary treatment through varying the inflation source and inflation means for the treatment apparatus or device.

A need exists for a system in which a compression garment includes a universal connector to a variety of pumps. Thus, the user could interchange between an intermittent pneumatic pressure pump, for instance when seated, and then change to a set pressure pump for instance when walking. The various type of pumps are not limited to the two listed above but could be any type of pump with a universal connector.

Further a need exists for a system in which a multiple of compression garments have separate active pressures. For instance, the inventive thigh compression garment could be used in conjunction with a lower leg compression apparatus such as that described (and incorporated herein as reference) in U.S. Pat. Nos. 9,033,906 and 7,967,766 and 7,559,908 and U.S. Ser. No. 13/444,600 and therefore the user could wear two separate therapeutic compression apparatus on the entire leg and could also include an optional knee wrap. In this instance the user could have one pressure level on the inventive thigh therapeutic compression apparatus whereas a calf compression garment could have a second pressure level and both compression garments are connected to one pneumatic pump configured to have multiple pressure outlets. Such a need exists for such a system.

Yet another need exists for compression on the arm where access is placed for dialysis treatments. Hemodialysis access for a patient undergoing dialysis typically via a fistula, graft, catheter, or stent usually on the arm. An artery and a vein in the arm is joined by a U-shaped plastic tube under the skin and thereafter needles are inserted into the graft when having each dialysis event. A fistula may be placed on the arm or hand for access during dialysis treatments. In other instances a venous catheter may be placed in the arm or a central venous catheter may be tunneled under the skin and placed in a vein in the neck, chest, or groin. Each of these may lead to edema, lymphedema, CVI or DVT in the arm, hand, neck, chest or groin. Thus, a need exists for a compression apparatus which can reduce lymphedema, CVI or DVT but is easier for the user undergoing dialysis treatments to place on the body part or which may be used as a preventive measure to stop the lymphedema, CVI or DVT from occurring.

A further need exists for a compression apparatus which can enhance exercise and recovery time by dealing with lactic acid build up in the muscles. A compression apparatus may enhance pre-exercise warm up by increasing the temperature and literally warming up the target muscles on the leg, calf, thigh, arm, shoulder, torso, chest and other body parts. A compression apparatus may assist recovery post-exercise by helping the blood remove the lactic acid. Reducing the lactate acid during exercise increases the lactic threshold of the muscles thus they perform better without experiencing cramping. Thus a need exists or a compression apparatus or system that is easier to place on the target body part pre-exercise, remain on securely during the exercise and post-exercise which can be placed with only one hand or using two hands and remains securely on the body part without downward slippage on the body part, especially during movement and even light or strenuous exercise. Such a long felt need is met by the inventive therapeutic compression apparatus and system.

Other known problems with the current treatment apparatus and devices, bandages, stockings and hosiery is the requirement that a skilled care-giver apply the current treatment apparatus and devices, bandages, stockings and hosiery. Such a skilled care-giver may not be available to all patients, notably those without long-term care insurance or provided a skilled home-health aid. Yet another known problem is leakage of set compression within the treatment apparatus and devices, bandages, stockings and hosiery resulting in an ineffective treatment and ineffective apparatus or device and the like which may be rendered useless to the patient and user. A further problem with the current treatment apparatus and devices, bandages, stockings and hosiery is that the inflation means or source of compression is set up as either manual or mechanical or electrical and cannot be interchanged in that the inflation port or inflation means is not universal and interchangeable. Yet another problem with the current treatment apparatus and devices, bandages, stockings and hosiery is that the inflation means or source is either static or intermittent and again cannot be changed during the treatment with such apparatus or device. Yet another problem with the current treatment apparatus and devices, bandages, stockings and hosiery is that the apparatus and device, bandages, stocking and hosiery slips down the leg or arm due to gravity and/or walking or movement of the user. There is a long felt need for a compression apparatus which maintains stability on the target limb or body part while maintain sufficient compression or pressure.

The apparatuses, methods, assemblies and systems of the subject invention provide benefits and advantages that may overcome a number of problems with respect to known compression technologies, particularly the problems that arise due to the difficulty of applying current compression wrap technologies. The subject invention provides an alternative to known technologies that employ tight-fitting therapeutic elastic garments, which cause patients discomfort, and lose their elasticity as well as have slippage down the leg, and therefore their effectiveness over time. Those skilled in the art will readily appreciate that it would be beneficial to provide a therapeutic compression device and system for treating CVI, DVT and lymphedema that is adapted and configured to apply localized compression to the leg, thigh, calf and/or foot to prevent swelling and further improve venous circulation, that may also be self-administered by a patient effectively.

Without limitation, the inventive therapeutic compression apparatus may be use with any required compression therapy, such as venous disease, vascular disease, lymphedema and the like. The inventive therapeutic compression apparatus may be used to treat any general swelling as well as being used post operatively for example including in cases of sclerotherapy or vein ablation. The inventive therapeutic compression apparatus may be used by a person for compression therapy such as athletes and lactic acid build up, or pregnant women, as well as any individual who walks a lot or is on their feet at work for period of time. Other uses for the inventive therapeutic compression apparatus may be envisioned.

The inventive therapeutic compression apparatus has an advantage of ease of use by the patient or user. The inventive therapeutic compression apparatus can be more easily applied onto the limb. The inventive therapeutic compression apparatus includes an elastic stocking or sleeve integral with an air bladder having variable compression when in use on the limb of the patient or user. Other advantages will be described for the inventive therapeutic compression apparatus.

### SUMMARY OF THE INVENTION

The subject invention is directed to a therapeutic compression apparatus and methods of use. The inventive therapeutic compression apparatus can be used on a limb such as a lower leg or a thigh or arm or any other limb of a user. The inventive therapeutic compression apparatus has n elastic stocking or sleeve that is connect to or integral with a wrap that together (or apart with just the elastic stocking or sleeve alone) are pulled on by the user over the target body part such as a leg or arm. Once puled to a sufficient location on the leg or arm or other body part, the wrap is closed around the target limb and then inflated to a chosen compression level, The inventive therapeutic compression apparatus may include a stabilization to stabilize the therapeutic compression wrap from slipping down the limb of the user when in use.

The therapeutic compression apparatus may further comprise at least one bladder operatively associated with the therapeutic compression apparatus for applying pressure to a treatment site on the limb such as a leg or arm. The therapeutic compression apparatus includes a connecting means such as a loop and hook material in order to wrap and close up the therapeutic compression apparatus around the thigh, calf, arm or other limb of the user. This connecting means or wrap may include at least one interior pocket for accommodating the at least one bladder. Alternatively, the at least one bladder may be integral with the connecting means or wrap. One or more connecting means may be operatively associated along the first and second peripheral edges of the therapeutic compression apparatus for securing it around the limb.

The securing means may be configured in one embodiment to be the elastic stocking or sleeve connect to the wrap by at least one button or other connecting means. In another embodiment the securing means is configured to have a belt inserted within the means of at least one aperture to stabilize the therapeutic compression apparatus on the user when in use. The securing means may also be configured as an adjustable strap around the waist, or hip, upper thigh area, calf, arm or another limb of a user.

The therapeutic compression apparatus may be formed at least in part of a non-elastic composite material comprising a plurality of distinct layers. In one embodiment, the composite material may comprise three distinct layers: an inner laminate layer, an outer hook-compatible layer, and a middle non-elastic layer provided between the inner and outer layers. The composite material may also be provided with a plurality of stitched darts and gathers for contouring the apparatus to the limb such as for instance the leg, knee, thigh, arm, shoulder or other area of the body. The wrap further includes a series of spot welds or linear welds placed so as to create the air bladder and compression profile onto the target limb or body part.

The subject invention is also directed to a bladder assembly for a compression apparatus for providing pressure to a limb. The bladder assembly comprises: at least one bladder having first and second flexible walls secured to one another about a peripheral edge thereof to form an air pocket; and at least one spot weld provided in a predetermined location inward of the peripheral edge connecting the first and second walls to one another to define a plurality of chambers within the bladder. The geometric placement of the at least one spot weld determines a gradient compression profile of the at least one bladder. The profile may be a gradient pressure profile and/or a gradient compression profile.

An inflation means for inflating the bladder such as the air pocket through at least one inflation port may be provided in the first wall of the bladder assembly. The inflation means may be detachable from the at least one inflation port. At least one pressure valve may be operatively associated with the inflation means for controlling an amount of pressure within the bladder and the air pocket within the bladder. The inflation port includes a check vale so as to maintain a given pressure within the bladder of the therapeutic compression apparatus. The inflation port may be universal in that it is configured to be capable of connecting to and accepting a plurality of inflation sources and inflation means such as a manual pump, mechanical pump, electrical pump, battery-operated pump, static pump, intermittent pump, pneumatic pump, negative pressure source and other variations.

A method of the invention includes the therapeutic treatment apparatus used to treat CVI, DVT and/or lymphedema by connecting the elastic stocking or sleeve to the wrap and then pulling up the elastic stocking or sleeve onto the target limb of the patient and then closing the wrap portion of the apparatus including the primary and secondary wraps around a limb by a patient and inserting an inflation means into an inflation port and inflating the bladders within the primary and secondary wraps and maintaining a certain pressure to treat the CVI, DVT and/or lymphedema.

Another embodiment of the present invention includes an assembly according to the invention includes a pressure mechanism having a flexible member for attachment to a limb and an air chamber which may be pumped up into a desired pressurized state, a separate relatively small pre-filled air bladder, an absorbent foam, sponge or dressing coupled to the pre-filled air bladder, and a suction conduit coupled to a source of negative pressure (suction) and in fluid communication with the absorbent foam, sponge or dressing. In a preferred embodiment, the pre-filled air bladder, the absorbent foam, sponge or dressing and the suction conduit are formed together as a unit.

According to one aspect of the invention, the flexible member of the pressure mechanism is adapted to wrap around a leg or arm and over the pre-filled air bladder in order to secure the pre-filled air bladder and the foam, sponge or dressing to a wound or ulcer in the extremity. Thus, the flexible member is provided with some fixation structure such as a hook and loop closure mechanism. An air pumping mechanism is preferably coupled to the air chamber of the pressure mechanism in order to inflate the air chamber to a pressurized state. The air chamber of the pressure mechanism is preferably designed to apply pressure along a predefined area (e.g., the saphenous vein of a leg) as opposed to around an entire limb.

According to another aspect of the invention, the suction conduit is located either between the pre- filled air bladder and the absorbent foam, sponge or dressing which is adhered to the small air bladder, or the pre-filled air bladder is formed as a donut with a central opening and the suction conduit extends through the central opening. By coupling the suction conduit to a source of negative pressure, exudate from the wound or ulcer is sucked through the foam, sponge or dressing into the suction conduit.

One of the methods of the invention include locating the pre-filled air bladder and foam, sponge or dressing over a wound or ulcer on a limb, wrapping the flexible member of the pressure mechanism around a limb with the air chamber located over the pre-filled air bladder/absorbent foam, sponge or dressing, and fastening the pneumatic pressure mechanism in place with the fixation structure. When the apparatus is properly located and affixed to the limb, the air chamber is inflated, preferably to 30-40 mm Hg, thereby applying pressure to the limb and more specifically via the pre-filled air bladder to the wound. The suction apparatus is activated by turning on the source of negative pressure, and exudate from the wound or ulcer is pulled through the absorbent foam, sponge or dressing into the suction conduit.

Another embodiment of the present invention includes an apparatus for applying intermittent pressure to a portion of the human body, such as an area of the human leg or arm, which assists with the healing and treatment of various conditions such as venous ulcers or wounds by promoting blood flow into and out of the area and by increasing drainage. The apparatus may include if on the leg a leg bladder, a calf bladder, a thigh bladder and/or a foot bladder and if on the arm a forearm bladder and/or an upper arm bladder, each having inflatable chambers that accommodate an entering fluid by inflating. The bladders are fluidly coupled by a fluid conduit, and each is preferably equipped with a means for locating it on a portion of the body. In a preferred embodiment, the leg bladder is positioned between the knee and ankle of the user and over the calf area. As a person walks while wearing the apparatus, a portion of the upper area of the leg bladder deflates as the person's leg moves foot (heel) strikes the ground due to the external pressure placed on the ankle area of the leg bladder, thereby adding more pressure therein. As the person's foot rolls from heel to toe in the standard walking motion, the external pressure from the person's weight affects the pressure within the air bladder. This process repeats as a person walks, thereby creating a pumping or kneading force on the leg as the pressure in the leg bladder intermittently increases and decreases, thereby promoting blood flow, fluid drainage, treatment, and healing to various parts of the leg.

In another embodiment for application to a thigh of a user, the thigh bladder is position between the pelvis or groin or hip area of the user and the knee of the user, and the apparatus further includes a calf bladder and a foot bladder, each having inflatable chambers that accommodate an entering fluid by inflating. The bladders are fluidly coupled by a fluid conduit, and each is preferably equipped with a means for locating it on a portion of the body. As a person walks while wearing the apparatus, a portion of the thigh bladder deflates as the person's leg moves foot (heel) strikes the ground due to the external pressure placed on the foot bladder, thereby forcing fluid out of the foot bladder, through the fluid conduit, and into the leg bladder, which raises the pressure therein. As the person's foot rolls from heel to toe in the standard walking motion, the external pressure from the person's weight is removed from the foot bladder, resulting in the pressure of the leg bladder being higher than the pressure in the foot bladder. Fluid thus flows back through the fluid conduit and into the foot bladder, which then inflates again to its original state, such that the pressures of the foot bladder and leg bladder are equalized. This process repeats as a person walks, thereby creating a pumping or kneading force on the leg as the pressure in the leg bladder intermittently increases and decreases, thereby promoting blood flow, fluid drainage, treatment, and healing to various parts of the leg.

In another embodiment including a therapeutic compression apparatus and an integral or separate lower leg therapeutic compression apparatus including a foot bladder, the foot bladder is positioned on a bottom of a foot and the leg bladder is positioned on a lower portion of a leg. As a person walks while wearing the apparatus, a portion of the foot bladder deflates as the person's foot (heel) strikes the ground due to the external pressure placed on the foot bladder, thereby forcing fluid out of the foot bladder, through the fluid conduit, and into the leg bladder, which raises the pressure therein. As the person's foot rolls from heel to toe in the standard walking motion, the external pressure from the person's weight is removed from the foot bladder, resulting in the pressure of the leg bladder being higher than the pressure in the foot bladder. Fluid thus flows back through the fluid conduit and into the foot bladder, which then inflates again to its original state, such that the pressures of the foot bladder and leg bladder are equalized. This process repeats as a person walks, thereby creating a pumping or kneading force on the leg as the pressure in the leg bladder intermittently increases and decreases, thereby promoting blood flow, fluid drainage, treatment, and healing to various parts of the leg.

In one embodiment of the present invention, the therapeutic compression apparatus includes a stabilization means such as at least one aperture configured to contain a belt or other means to secure the therapeutic compression apparatus to the user's waist when in use and stabilize the therapeutic compression apparatus to reduce slippage downward towards the knee. In another embodiment of the invention the stabilization means is a series of apertures on the top horizontal edge of the therapeutic compression apparatus configured to the attached to a hook or other connecting means to a garter belt or other means secured around the user's waist. Other stabilization means to reduce slippage of the therapeutic compression apparatus may be included.

In another embodiment of the present invention, the therapeutic compression apparatus includes two bladders, a lateral bladder and a medial bladder. The medial bladder has a gradient pressure profile based on the geometric location of the spot welds located on the medial bladder. The lateral bladder may be connected to the medial bladder and the lateral bladder also has a separate gradient profile based on the geometric location of the spot welds on the medial bladder. In another embodiment there is either no lateral bladder or the lateral bladder is closed off and noninflated. These and other aspects of the contacts of the subject invention will become more readily apparent from the following description taken in conjunction with the drawings.

### BRIEF DESCRIPTION OF THE DRAWINGS

So that those having ordinary skill in the art to which the subject invention pertains will more readily understand how to make and use the apparatuses of the subject invention, preferred embodiments thereof will be described in detail herein below with reference to the drawings, wherein:

FIG. 1 is a view of one embodiment of the present invention including a therapeutic compression apparatus configured to be placed on a lower leg and constructed in accordance with the present invention, showing the elastic sock and attached wrap from the front view and the optional compression strap for the foot;

FIG. 2 is another view of the embodiment of FIG. 1 from the back showing the buttons inserted into the apertures and connected to the wrap;

FIG. 3 is an exploded view of the embodiment of FIG. 2 from the back view and including the optional compression strap for the foot;

FIG. 4 is another embodiment of the present invention with a different configuration of spot welds and linear welds on the wrap creating a different gradient compression profile, showing the elastic sock and attached wrap from the front view;

FIGS. 5A-5C are various embodiments of the elastic sock with 5A including a loop to secure the optional compression strap for the foot, 5B including a compression portion on the foot and an adhesion connecting means for the wrap, and 5C including stud buttons as the connecting means to the wrap;

FIGS. 6A-6E is a method of use with 6A showing the user pulling the elastic sock (connected to the wrap) over the foot and up the calf, 6B showing the elastic sock placed on the lower leg with the proximal end portion near the knee, 6C showing the inventive therapeutic compression apparatus on the lower leg with the wrap in the closed position and being inflated via a hand pump connected to the inflation port to inflate the bladder, 6D is another embodiment of the method of use with the elastic sock alone placed and pulled up on the lower leg near the knee, and 6E showing the wrap being connected to the connecting means on the elastic sock;

FIG. 7 is another embodiment of the present invention including a therapeutic compression apparatus configured to be placed on an arm and constructed in accordance with the present invention, showing the elastic sleeve and attached wrap from the back showing the buttons inserted into the apertures and connected to the wrap;

FIG. 8 is an exploded view of the embodiment of FIG. 7;

FIG. 9 is a side view of the embodiment of FIG. 7 showing the therapeutic compression apparatus on the arm of a user;

FIG. 10 is an embodiment of the present invention showing a belt system, automatic pump and hoses connected to a lower leg embodiment of FIG. 1 and an arm embodiment of FIG. 7;

FIG. 11 is an embodiment of FIG. 10 shown on the body of a user and including a hand pump; and

FIG. 12 is another embodiment of the present invention including a therapeutic compression apparatus configured to be placed on a thigh and constructed in accordance with the present invention, showing the elastic sleeve and wrap from the front in the open and non-connected position.

### DETAILED DESCRIPTION OF THE PREFERRED EMBODIMENTS

Preferred embodiments of the subject invention are described below with reference to the accompanying drawings, in which like reference numerals represent the same or similar elements. One of ordinary skill in the art would appreciate that while the apparatuses discussed herein relate to compression therapy of the leg, the scope of the invention is not limited to those exemplary applications and may be sized and shaped for the anatomical portion for which compression therapy is needed including without limitation the arm, torso, etc.

The subject invention provides compression to a patient's limbs, including the extremities, including for example, the leg of a user and more specifically the calf or thigh of a user, or an arm of a user, in a manner that is simpler, easier, more convenient and more secure than current systems. Any limb or body part may be compressed by the instant therapeutic compression apparatus such as for instance a foot, calf, thigh, knee, leg, hip, buttocks, waist, torso, ribs, shoulder, arm, hand, fingers, neck, head or the like.

The subject invention provides system for providing compression and preventing swelling of a limb such as for instance the calf, thigh or arm using an elastic sleeve connected to a wrap including a compression wrap having a non-elastic binder and bladder, which can be used for compression. The elastic sleeve or sock may have a compression element as well in addition to the compression wrap. The bladder is provided within a non-elastic wrap and creates compression in a manner that allows for consistent measuring of the pressure supplied, as well as safe, comfortable, convenient, effective, self-application by the patient.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” would include a plurality of such stimuli and reference to “the signal” would include reference to one or more signals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

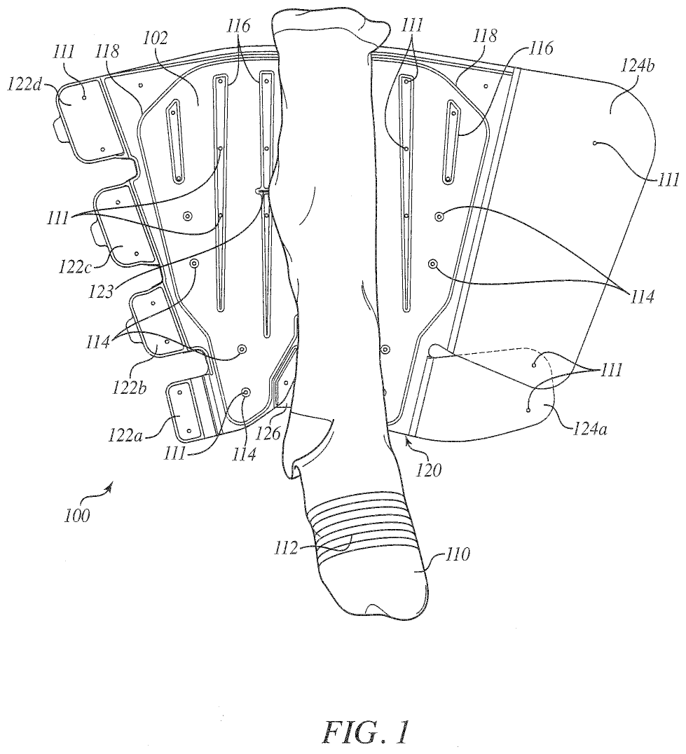

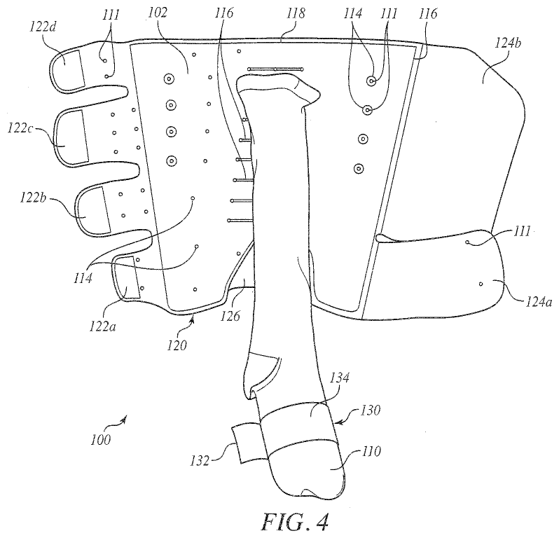

Referring now to FIG. 1, there is illustrated an exemplary embodiment of a therapeutic compression apparatus 100 according to the subject invention showing one embodiment of the present invention being a lower leg therapeutic compression apparatus 100 including an elastic sleeve or elastic sock 110 connected to a wrap 120 together integrally formed in the therapeutic compression apparatus 100. The therapeutic compression apparatus 100 is configured and adapted to wrap around a patient's limb such as in this instance as shown in FIG. 1 a lower leg, though it could be used in other limbs such as for instance a thigh, torso, chest, arm, upper arm, lower arm, forearm, etc. The therapeutic compression apparatus 100 has an elastic sock or sleeve 110 connected to a wrap member 120 with a proximal end portion (top as oriented in FIGS. 1-6) and opposed distal end portion (bottom as oriented in FIGS. 1-6) which in this embodiment is configured and adapted to conform around a patient's lower leg and provide compression through the inflation of bladder 102. In another embodiment not shown, compression is created by closing the wrap 120 onto the target body part and optionally tightening the fasteners such as for instance loop 124 and hook 122 to create higher compression by tightening the connection.

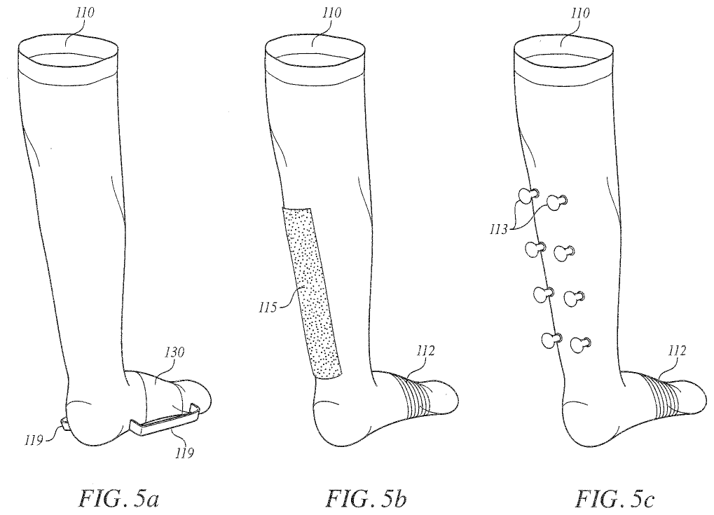

The sock or sleeve 110 is elastic and can include compression throughout, or only a portion having a compression element, or be non-compression. The elastic sock or sleeve 110 may be comprised of materials such as nylon, wool, cotton or any other compatible elastic material. The elastic sock or sleeve 110 may have a feature of compression itself which is then another level of compression coupled with the wrap 120 and/or compression or pressure of the bladder 102 once inflated. In one embodiment as shown in FIGS. 1, 3, 5B-5C and 6A-6E, the foot portion of the elastic sock or sleeve includes a compression portion 112 located around the arch and sole of the foot portion. An optional compression strap 130 may be included which has a loop portion 134 and a hook portion 132 to secure the compression strap together. Other known connecting means for the compression strap 130 may be included such as without limitation VELCRO™, hooks, snaps, adhesion, sewing, buttons, or the like. The compression strap 130 may be secured on the foot located to encircle the arch and sole of the foot or it could be secured above or below the ankle or knee or any other body part to create compression on the target body part. FIG. 5A includes an embodiment of an elastic sock or sleeve 110 which has at least one, in this embodiment a pair, of loops 119 on each side of the middle foot so that the compression strap 130 can be inserted within each loop 119 to further secure the compression strap 130 onto the foot portion of the elastic sock or sleeve 110. In other embodiments not shown, the compression strap may be larger to create targeted compression onto a wrist, elbow, shoulder, bicep, triceps, waist, torso, chest, thigh or any other body part used simultaneously with any of the therapeutic compression apparatus 100, 200, 300 on any body part. In other embodiments not shown the elastic sock 110 may not include a foot portion but have openings on each of the top and bottom portion of the elastic sock 110, or have an ankle portion or heel portion but not a closed toe portion or any combination thereof.

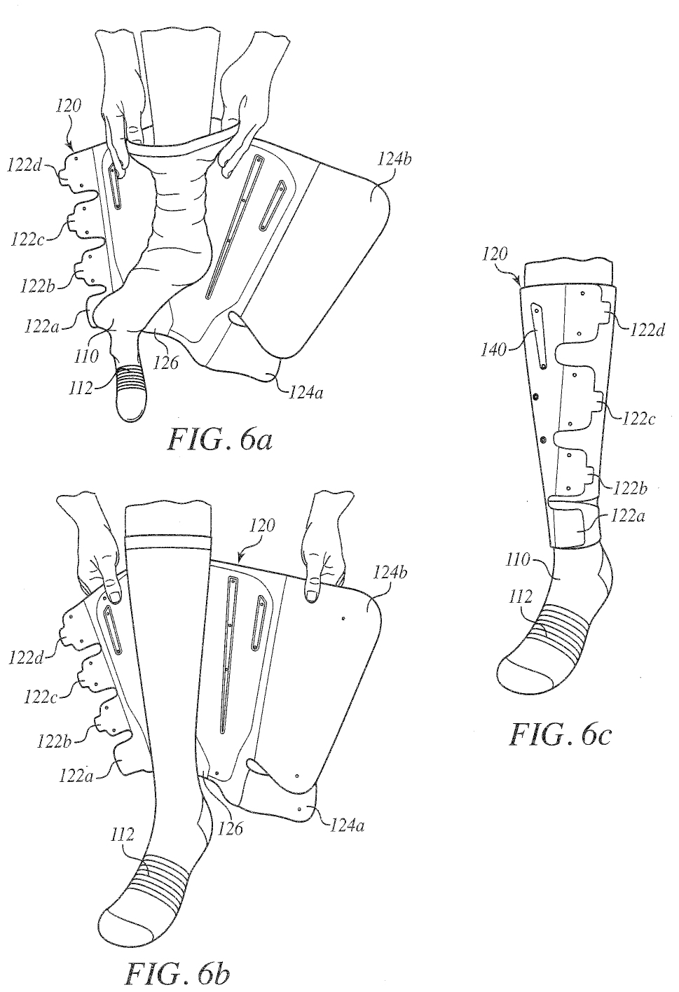

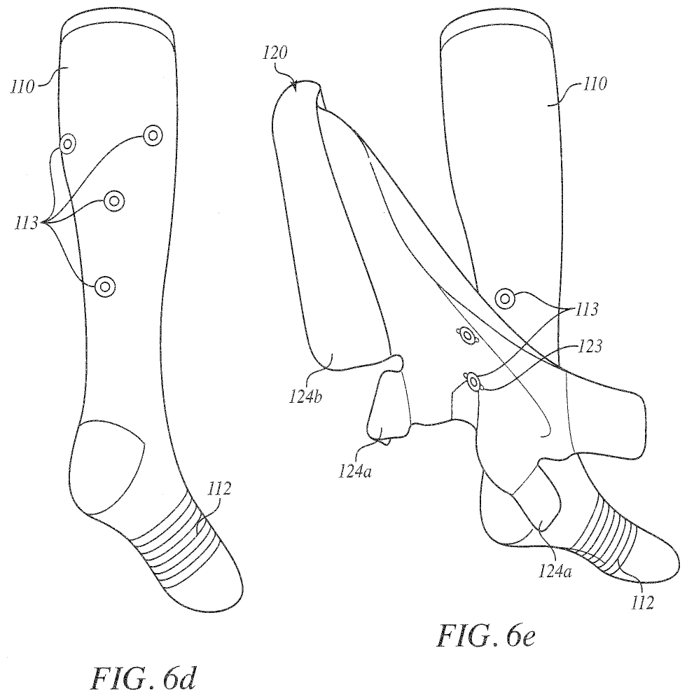

The elastic sock or sleeve 110 is connected via a connecting means to the wrap 120. In one embodiment shown in FIGS. 5A-5C, the connecting means may be via adhesion, sewing, welding, gluing, snaps, hooks, buttons, Velcro™ or any other known connecting means. FIGS. 1-4, as well as FIGS. 6A-6E, show the connecting means as buttons 113 located on the elastic sock or sleeve 110 (210 and 310 in the arm and thigh embodiments) which can be inserted and secured within slits 113 (223 and 333) on the wrap 120 (220 and 320). The number of connecting means 113 (213, 313) can be at least one but may be in an amount sufficient to connect the elastic sock or sleeve 110 (210, 310) to the wrap 120 (220, 320). The area 115 of FIG. 5B of elastic sock or sleeve 110 is connected or joined to the area (not shown)on the wrap 120 located above the heel portion 126. Other embodiments could include VELCRO™ or loop and hook features. In another embodiment as shown in FIG. 5C, the connecting means is a set of stud buttons 123 located on the elastic sock or sleeve 110 which mates to the female portion opening slit 123 located on the wrap 120. The stud buttons 113 of FIG. 5C have a diameter small enough to be placed within the slit 123 but large enough so than when moved into the closed position secure the elastic sock or sleeve 110 to the wrap 120. In an embodiment not shown the stud button 113 could be a washer or other element which connects or snaps into an opening to connect and secure elastic sock or sleeve 110 to the wrap 120. In most of the embodiments shown here based on ease of manufacturing, cost and use by the individual user, the connecting means is a set of buttons wherein a male portion of the button 113 (213, 313) is located on the elastic sock or sleeve 110 (210, 310) which mates to the female opening portion 123 (223, 323) of the button 123 located on the wrap 120. In this embodiment the female opening portion 123 (223, 323) is a slit aperture but it could be any other type of aperture or if the connecting means was a snap it would be the combination of the female and male portions of the snap. Other connecting means could include snaps, hooks, buckles, straps, zippers and other combinations of known connecting means.

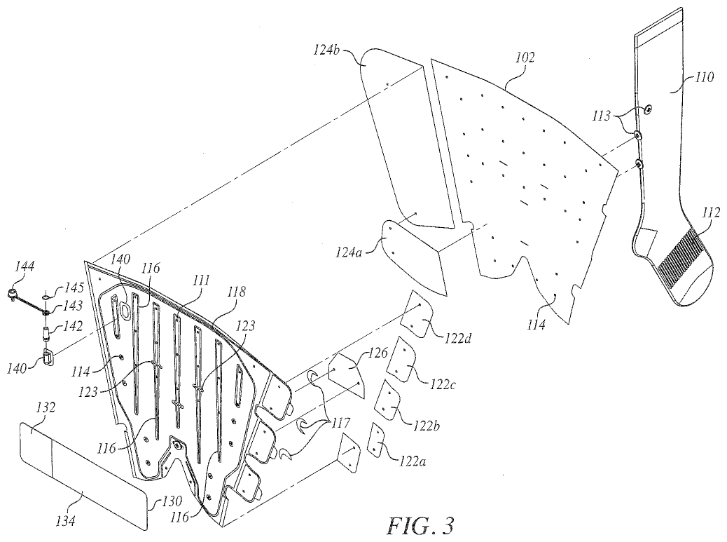

Bladder 102 includes an inner sheet and outer sheet made out of a nylon laminated polyurethane sheet which are configured and adapted to be RF welded together as shown in FIG. 3. However, any other suitable materials which are weldable or otherwise joined while being airtight can be used. In this exemplary embodiment, bladder 102 is a single continuous bladder throughout however, it is envisioned that the therapeutic compression apparatus 100 could have an independent bladder either separately inflatable or inflatable through a one-way valve or other desired inflation/deflation configuration. Non-limiting examples of bladders 102 and wraps 120 can be found in co-owned patent application Ser. No. 16/846,211 and PCT/US20/31227 which are hereby incorporated by reference herein in their entirety. In an embodiment now shown, the wrap 120 does not include a bladder and the compression element is created by the closure of the wrap such as by connecting the hook and loop fasteners 122, 124 (222, 224, 322, 324) with higher compression created by tightening the fasteners 122, 124 (222, 224, 322, 324)

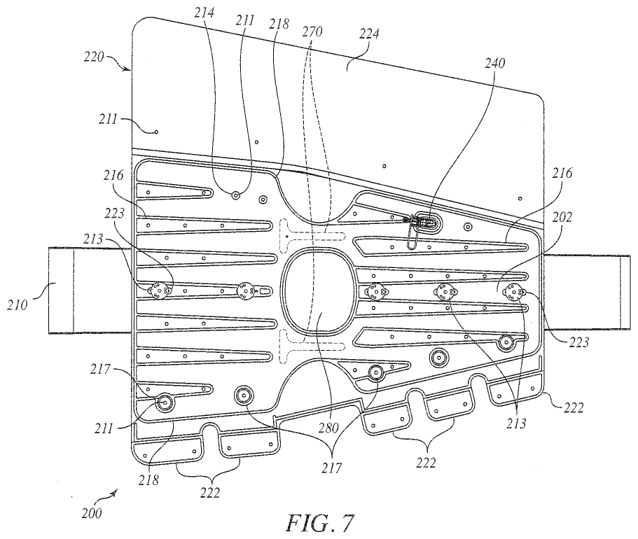

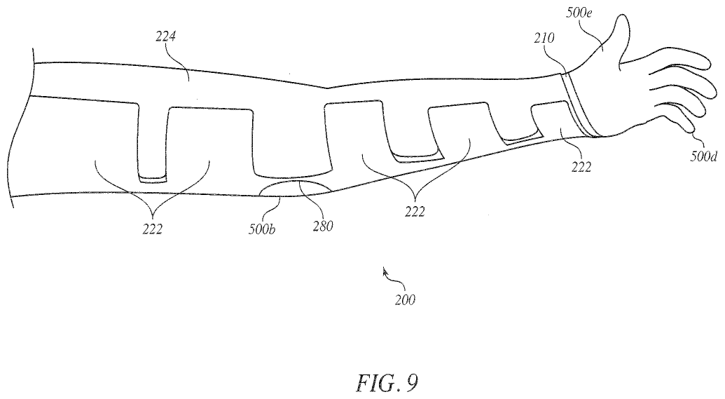

In this embodiment as shown in FIGS. 1-6E, hook and loop fasteners 122, 124 (222, 322, 224 and 324 in the arm and thigh embodiments shown in FIGS. 7-12) are provided along the edge of inner and outer sheets in order to ease adjustment and secure therapeutic compression apparatus 100 (200, 300) on a patient's limb such as for example a lower leg or calf as shown in FIGS. 1-6. It is envisioned that the therapeutic compression apparatus 100 can also be secured to a patient's limb by other means, such as zippered, buttoned, buckled, strapped, or be cuff shaped by other such suitable means. Further, it is also envisioned that hook and loop closures 122, 124 can be replaced by material similar to that of strap described below and be welded/sewn/attached to wrap 120 for improved comfort.

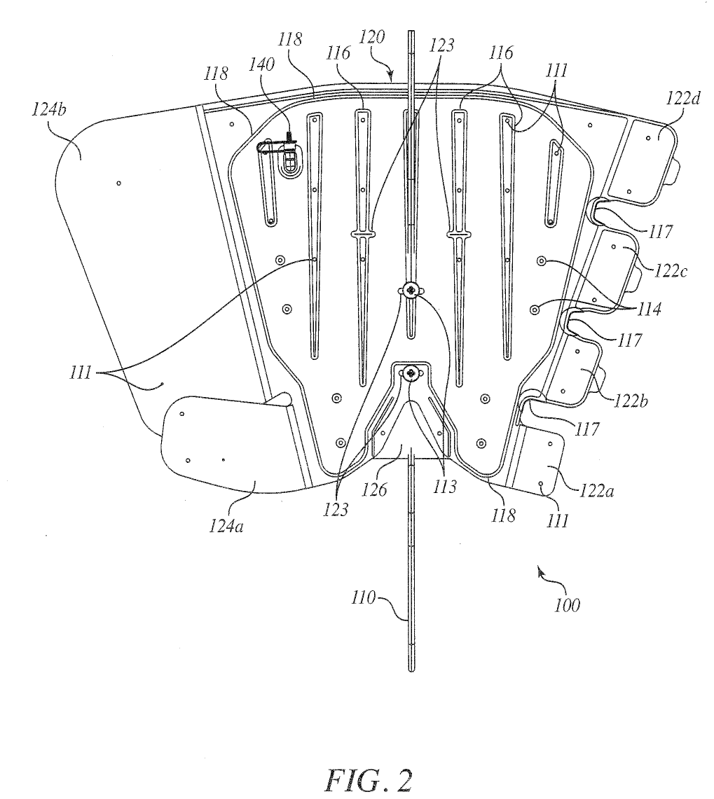

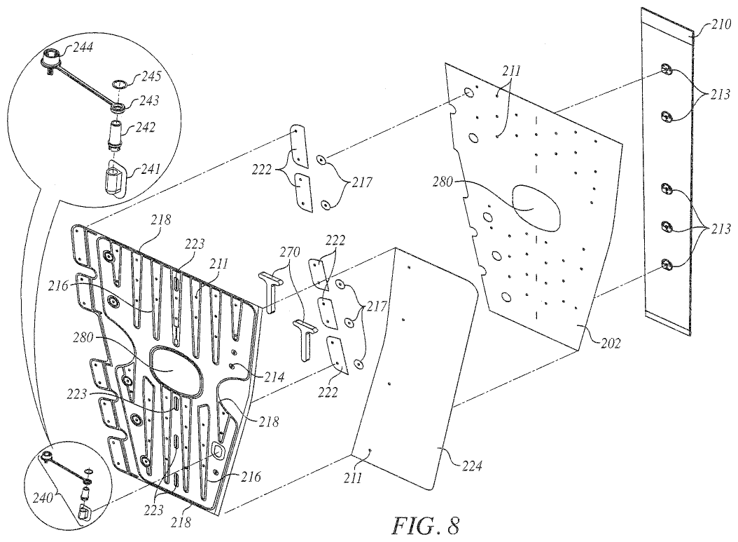

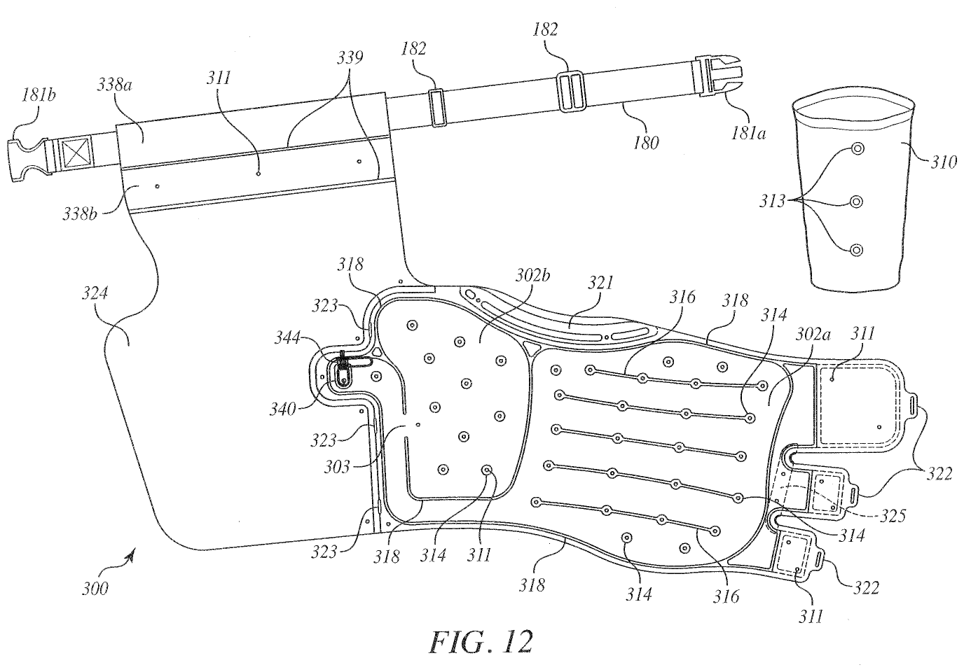

Further, optional supports may be included to reduce tearing of the wrap 120 or hook 122 (222, 322) when pulling the hook portion 122 (222, 322) off the loop portion 124 (224, 324). As seen in FIGS. 2-3, a series of strain relief disks 117 are included between each of the hook portions 122a, 122b, 122c and 122d to reduce tearing of the material after repeated use by the individual. Optional strain relief disks 217 are also shown in FIGS. 7-8 on the wrap 220 again to reduce tearing of the material by the user when opening the wrap 220 and pulling on the hook portions 222 off of the loop portion 224. Finally, an optional strain relief 325 is shown in FIG. 12 which may be a connector to strengthen the area between the hook fastener 324 to prevent tearing of the material when in constant use by the individual.

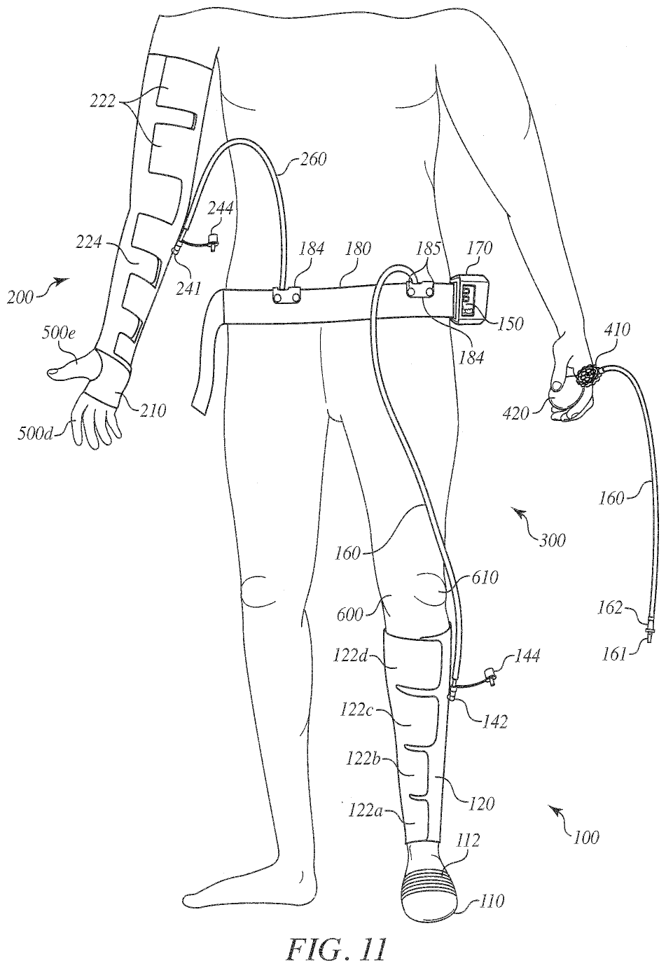

In this lower leg therapeutic compression apparatus embodiment 100 and in the arm and thigh embodiments (200, 300) inflation means may be a device such as a hand pump 400 (as shown in FIG. 11 having a bulb 420 and hand dial 410) capable of attaching to inflation port 140 (240, 340) to inflate bladder 102 (202, 302). The inflation port 140, 240, 340 includes a female luer 142, 242, 342 within inflation port housing 140, 240, 340, and a cap 144, 244, 344 which is connected to the luer 14, 242, 342 via a connector ring 143, 243, 343. The cap 144, 244, 344 closes the inflation port 140, 240, 340 and is sealed with a ring 145. 245. 345. Other types of inflation ports may be employed.

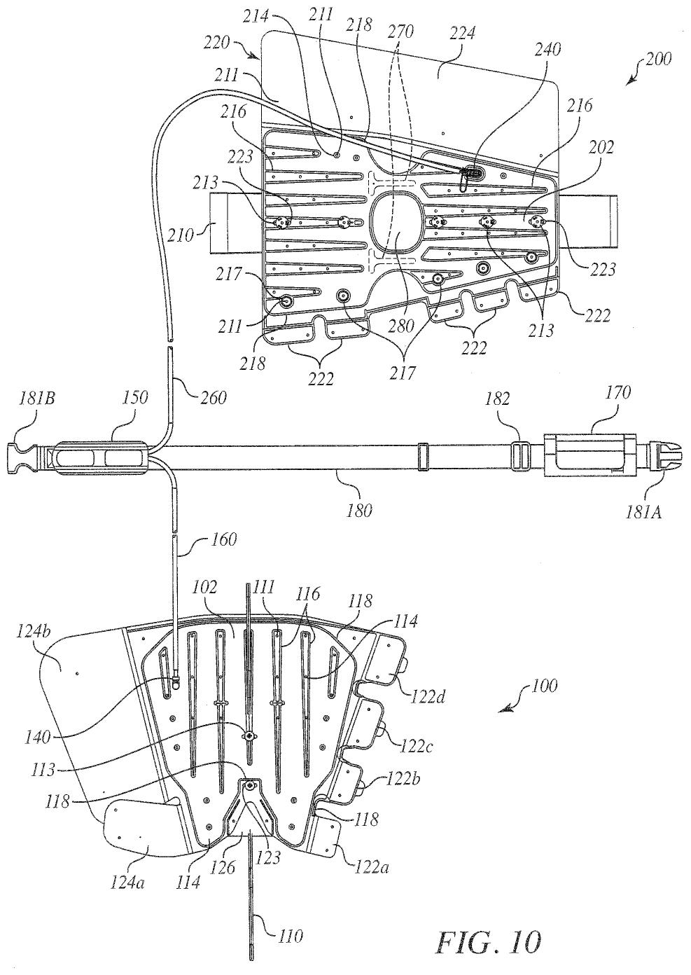

It can be appreciated that a mechanical or automatic inflation pump 150 (as shown in FIGS. 10-12) can also be attached to inflation port 140 (240, 340) to inflate and deflate bladder 102 (202, 302) to provide pulsating pressure to a user's limb such as a calf or lower leg or a thigh or an arm. As shown in FIG. 11, the inflation means to connect to the inflation port 140, 240, 340 is a hand manual pump 400 including a bulb 420 for manually pumping of air fluid and a hand dial 410 including a check valve within. A number or variety of inflation means can be employed in any of the therapeutic compression systems and apparatus 100, 200, 300 such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a certain pressure within the bladder so to provide compression in use by the patient. A vent valve can also be incorporated into therapeutic compression apparatus 100, 200, 300 or with inflation means to allow a user to selectively deflate bladder 102. Further, a check valve or relief valve is incorporated with either inflation means or bladder 102 to prevent over-inflation once a maximum pressure is detected. Examples of relief valves are described in U.S. Pat. Nos. 7,276,037 and 7,850,629, the disclosures of which are incorporated by reference in their entirety as well as the disclosure of the above-referenced co-owned patent applications and issued patents.

In this embodiment of the therapeutic compression apparatus 100 for application to a user's lower leg, the therapeutic compression apparatus 100 includes a panel 126 located along the distal end portion of the therapeutic compression apparatus 100 configured to conform to the ankle area of the leg. The panel 126 may be comprised of a flexible material such as but not limited to nylon, polyurethane, cotton, or other suitable material, or a material such as Spandex. The panel 126 may be configured to prevent the occurrence of a pinch point and reduce pressure on a patient's ankle area when wearing therapeutic compression apparatus 100.

In another embodiment not shown, the therapeutic compression apparatus may include an optional knee pad which may be separate or connected to the therapeutic compression apparatus 100. The optional knee pad can be connected to the therapeutic compression apparatus 100 though a connecting means of located on the optional knee pad or an aperture of other married connecting means located on the therapeutic compression apparatus 100. Any other connecting means may be employed such as but not limited to hook and loop closure or VELCRO™ or any other known connecting means such as buckles, straps, buttons, snaps, zippers and other combination. In the alternative the connecting means may be welded together or adhered or other connecting manner. The optional knee pad may be comprised of a flexible material such as but not limited to nylon, polyurethane, cotton, or other suitable material, or a material such as Spandex. Another optional pad could be a groin pad if the therapeutic compression apparatus 100 is applied to the user's thigh. Other optional pads may be employed for the comfort of the patient and to reduce slippage of the therapeutic compression apparatus 100 when in use and the user walks or moves about.

Bladder 102 (202, 302) can be integral within inner sheet and outer sheet, the location and desired preconfigured compression gradient profile can be obtained cost-effectively. A number of different embodiments of bladder configurations can be used in the therapeutic compression apparatus 100 (200, 300) of the subject invention such as those configurations described above. In another embodiment not shown the wrap 120 does not have any bladder(s). FIGS. 1-6 show therapeutic compression apparatus 100 having bladder 102 with a plurality of spot welds 114 and linear welds 116 therein. FIGS. 7-9 show therapeutic compression apparatus 200 having bladder 202 with a plurality of spot welds 214 and linear welds 216 therein. FIG. 12 shows therapeutic compression apparatus 300 having bladder 302 (and portions 302a, 302b) with a plurality of spot welds 314 and linear welds 316 therein. Each of spot welds 114, 214, 314 and/or linear welds 116, 216, 316 are strategically placed within bladder 102, 202, 302 (302a, 302b) in a predetermined pattern based on the desired gradient profile relative to the compression needed at the patient's treatment site. Spot welds 114, 214, 314 and/or linear welds 116, 216, 316 enable bladder 102, 202, 302 (302a, 302b) to define the gradient profile when inflated through inflation port 140, 240, 340. The geometric placement of spot welds 114, 214, 314 and/or linear welds 116, 216, 316 within bladder 102, 202, 302 (302a, 302b) allows increased inflation of certain portions of bladder 102, 202, 302 (302a, 302b), and can create one or more fluid chambers within bladder 102, 202, 302 (302a, 302b). This configuration is particularly useful when compression is needed to improve fluid movement (e.g., blood, lymph, etc.) within the body. Further, linear weld lines 116, 316, allow for better compression along the calf in one embodiment or inner thigh and outer thigh of a patient's thigh by increasing tension applied to the medial portion of the thigh of a patient in another embodiment. This increased tension created by any of the spot welds 114, 214, 314 and linear welds 116, 216, 316 can generate a more effective compression in order to increase venous flow to the target limb or body part. For example, linear welds 316 in FIG. 12 located laterally along the medial portion of the thigh create a ribbed portion, which keeps the inflated profile of therapeutic compression apparatus 300 compact which can further increase ambulation and reduce interference with a patient's clothes.

It can be appreciated that depending on the location of the therapeutic compression apparatus, different pressure gradients may be utilized. Other possible gradient pressure profiles may be imagined based upon the geometric location of the spot welds 114, 214, 314, alone or in connection with the linear welds 116, 216, 316.

Referring now to FIGS. 1-6. therapeutic compression apparatus 100 is connect by the connecting means, in one example via the buttons 123 inserted into the female opening 123, thus connecting the elastic sock or sleeve 110 to the wrap 120. As shown in FIG. 6A, the user then places the elastic sock or sleeve 110 over the foot and pulls the entire therapeutic compression apparatus 100 up the calf to the desired location on the target limb as shown in FIG. 6B, which may be the lower leg or calf. The user then easily closes the wrap 120 via the hook and loop 122, 124, as the wrap is located securely and with stability on the target body part, which can be done by connecting or placing the wrap into the closed position by connecting hook 122a to loop 124a and then hooks 122b, 122c, and 122d to loop 124b in any order, such as lower to higher or higher to lower. Once the wrap is in the closed position as shown in FIG. 6C, the user then inflates the bladder 102 so that the therapeutic compression apparatus 100 is not able to shift out of place, thus increasing comfort and reducing fitting issues on the patient. In the alternative, the user can pull the elastic sock or sleeve 110 up on the lower leg as shown in FIG. 6D and then connect the wrap 120 via the connecting means, in this embodiment the buttons 113 on the elastic sock or sleeve 110 into the apertures 123 on the wrap 120. The therapeutic compression apparatus 100 is easier for the patient to apply or place on the lower leg as it is one integral apparatus in the embodiment shown in FIGS. 1-6. The user, notably a user with reduced mobility in his or her hands, legs or back, can more easier place the inventive therapeutic compression apparatus 100 on the leg without need for a skilled care-giver to assist. Further, the therapeutic compression apparatus 100 remains secure on the lower leg and has decreased slippage down from the knee to the ankle portion compared to compression stockings or other wraps.

In accordance with another exemplary embodiment not shown, inner sheet further includes a layer that has a first elastic modulus, inner sheet has a second elastic modulus. The first elastic modulus is less than the second elastic modulus in a transverse direction relative to the proximal and distal end portions of therapeutic compression apparatus 100, 200, 300 to wrap therapeutic compression apparatus 100, 200, 300 around the leg, calf, thigh or arm (or any other body part or limb) when the compression bladder 102, 202, 302 is inflated. In an exemplary embodiment, inner sheet includes a secondary sheet disposed on an inner surface thereof, to directly contact the calf, lower leg, thigh or arm (or any other body part or limb) in use. The secondary sheet can be a fabric layer, which is elastic in a first direction and inelastic in a second direction to curl the wrap member around the leg, calf, thigh or arm (or any other body part or limb) when the compression bladder 102, 202, 302 is inflated.

In another exemplary embodiment, a hip strap (not shown) or a waist strap (not shown) is configured and adapted to improve wearability of therapeutic compression apparatus 100, 300 by locating a portion of bladder 102 above the widest portion of the thigh of a patient and provides stability of therapeutic compression apparatus 100, 300 by preventing therapeutic compression apparatus 100, 300 from slipping down the thigh of a patient towards the knee and increase stability and improving thigh compression.

It will be appreciated that the chambers of the bladder 102, 202, 302 may be filled by air, fluid or other known means of inflation. It will also be appreciated that the bladder 102, 202, 302 can be arranged to receive air and be inflated using a manual pumping bulb 420, or can be inflated by an electric air pump 150 which can use batteries or AC wall current to pump air into the chamber(s). Any known source of air or fluid may be employed whether manual, mechanical, electrical, battery-operated or any other power sourced pump or pressure creator. The inflation means may be a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a certain pressure within the bladder so to provide compression in use by the patient. As shown in FIGS. 10-12, a belt system 180 may be employed which includes an automatic pump 150 and a pump cover 170 configured to be placed over the automatic pump 150. In this embodiment, the retaining means 180 is a belt or strap and includes a belt snap 181A (male connector) and a belt clip 181B (female connector), which are joined together by the belt strap 181B being inserted within the belt clip 181A and then snapped together. Other embodiments not shown may include a garter belt system or a belt buckle or any other known closing or connecting means. Again, the retaining means 180 if a belt may be closed or connected though other connecting means such as for example and not limited to hook and loop closure or VELCRO™ or any other known connecting means such as buckles, straps, buttons, snaps, zippers, hooks and other combinations. As shown in FIGS. 11-12, the retaining means 180 belt is worn at the waist of the patient. In another exemplary embodiment, the retaining means 180 may be a hip strap (not shown) or a waist strap (not shown as what is shown is a belt embodiment) is configured and adapted to improve wearability of the inflation means 150 and increased ambulation for the patient. An optional protective cover is a flexible cover 170 so to protect the inflation means 150 when dropped by the user or just common wear and tear on the pump mechanism or even to prevent over heating or chaffing at the waist of the user. The flexible cover 170 may be comprised of a polymer, rubber or other flexible materials or the like.

It will be appreciated that the chambers of the bladder 102, 202, 302 may be filled by air, fluid or other known means of inflation, It will also be appreciated that the bladder 102, 202, 302 can be arranged to receive air and be inflated via the inflation port 140, 240, 340 using a manual pumping bulb or can be inflated by an electric air pump which can use batteries or AC wall current to pump air into the chamber(s). The inflation port 140, 240, 340 of the therapeutic compression apparatus 100, 200, 300 is universal in that it can connect to many different types of inflation means.

As shown in FIGS. 10-12, in these embodiments the inflation means 150 is connected to the inflation port 140, 240 via a tube 160, 260 or hose. Any other known connecting means may be employed. The embodiments shown include a clear tube 160 connected to the hand held manual pump 400 and another embodiment of the tube 160 is shown with a covering 162 on the tube, shown as black cloth 162 so as to allow the user to wear the covered tubes 160 more easier under clothing or less likely to be seen when worn over clothing. The tube 160 is connected to the therapeutic compression apparatus 100, 200, 300 by inserting, for instance a male luer slip 161, into the inflation port 140, 240, 340 within female luer 142, 242, 342. At the opposite end of the tube 160, the tube 160 is inserted into the inflation means, via the pump output port(s) 152. The hose 160 may be included within the belt 180 portion and reinforced by rivets 185 (or other means) and cover 184.

It can be appreciated that depending on the location of the therapeutic compression apparatus 100, 200, 300 being placed on the patient's body part or limb, different pressure gradients may be utilized. Further, different pressure gradients may be employed depending on the treatment (general swelling, lactic acid build up, lymphedema, post-sclerotherapy, CVT, DVT, etc.) and the treatment site. Other examples of bladder pressure gradient profiles are described in U.S. patent application Ser. No. 12/911,563 and U.S. patent application Ser. No. 12/855,185, the disclosures of which are incorporated by reference in their entirety.

In order to improve comfort, through-holes 111, 211, 311 are incorporated into the spot welds 114, 214, 314 and within the non-bladder portions of the wrap 120, 220, 320 between the linear welds 116, 216, 316. The through holes 111, 211, 311 may be located throughout therapeutic compression apparatus 100, 200, 300 in order to allow for ventilation about a patient's leg, thigh, arm or other body part or limb during extended wear of therapeutic compression apparatus 100, 200, 300. For the sake of clarity, not all of the through-holes are identified with reference characters in the Figures.

Another embodiment of the present invention includes a therapeutic compression apparatus 200 capable of being secured on the arm 500 of a user. The arm therapeutic compression apparatus 200 according to the subject invention includes an elastic sleeve or elastic sock or sleeve 210 connected to a wrap 220 together integrally formed in the therapeutic compression apparatus 200. The therapeutic compression apparatus 200 is configured and adapted to wrap around a patient's limb such as in this instance as shown in FIG. 7-9 an arm 500 from the shoulder 500a to the wrist 500c, though it could be used in other limbs. The therapeutic compression apparatus 200 has an elastic sleeve 210 connected to a wrap member 220 with a proximal end portion located nearer the shoulder 500a (wider portion which as oriented on top in FIGS. 8 and 11 and to the left as oriented in FIGS. 7 and 10) and opposed distal end portion nearer the wrist (thinner portion as oriented on bottom in FIGS. 8 and 11 and to the right as oriented in FIGS. 7 and 10) which in this embodiment is configured and adapted to conform around a patient's arm and provide compression through the inflation of bladder 202.

FIGS. 7-11 include another embodiment of the present invention of an arm therapeutic compression apparatus 200 including an elastic sleeve 210 integral and connected to a wrap 220. The elastic sleeve 210 is elastic and can be compression or non-compression. The elastic sleeve 210 may be comprised of materials such as nylon, wool, cotton or any other compatible elastic material. The elastic sleeve 210 may have a feature of compression itself which is then another level of compression coupled with the pressure of the bladder 202 once inflated. The elastic sleeve 210 may be open on both the proximal end nearer the shoulder and the distal end nearer the wrist or it may be closed at the distal end such as a mitten to be placed over the hand 500D or with finger portions and a thumb 500E potion such as a glove. In another embodiment of the elastic sleeve 210 the distal end may be open with an optional aperture configured to insert a thumb 500E and better secure the elastic sleeve 210 to the arm 500 of the user, as shown in FIG. 11. The elastic sleeve 210 is connected via a connecting means to the wrap 220. The connecting means may be via adhesion, sewing, welding, gluing, snaps, hooks, buttons, Velcro™ or any other known connecting means.

In this embodiment of the therapeutic compression apparatus 200 for application to a user's arm 500, the apparatus 200 includes an opening or aperture 280 located to correspond with the user's elbow 500b. An option panel (not shown) may be included configured to conform to the shoulder area 500a, elbow area 500b or wrist area 500c. The optional panel(s) may be comprised of a flexible material such as but not limited to nylon, polyurethane, cotton, or other suitable material, or a material such as Spandex. The optional panel(s) may be configured to prevent the occurrence of a pinch point and reduce pressure on a patient's shoulder area 500a, elbow area 500b or wrist area 500c when wearing the therapeutic compression apparatus 200. Other optional panels or pads may be employed for the comfort of the patient and to reduce slippage in a downward direction towards the wrist 500c or hand 500d of the therapeutic compression apparatus 200 when in use and the user walks or moves about.

The bladder 202 has a plurality of linear welds 216 and a plurality of spot welds 214 therein. Each of the linear welds 216 and spot welds 214 are strategically placed within bladder 202 in a predetermined pattern based on the desired gradient profile relative to the compression needed at the patient's treatment site. The geometric placement of linear welds 216 and spot welds 214 within bladder 202 allows increased inflation of certain portions of bladder 202, and can create one or more fluid chambers within bladder 202. This configuration is particularly useful when compression is needed to improve fluid movement (e.g., blood, lymph, etc.) within the body. This increased tension can generate a more effective compression in order to increase venous flow on the arm. Linear welds 216 may create a ribbed portion, which keeps the inflated profile of therapeutic compression apparatus 200 compact which can further increase ambulation and reduce interference with a patient's clothes. It can be appreciated that depending on the location of the therapeutic compression apparatus, different pressure gradients may be utilized. Other possible gradient pressure profiles may be imagined based upon the geometric location of the spot welds 214, alone or in connection with the linear welds 216. The gradient compression profile may be different depending on the treatment whether for lymphedema, dialysis, DVT or other uses of the therapeutic compression apparatus 200.

The arm therapeutic compression apparatus 200 may optionally include an expander 270 such as by way of example only a foam expander. The optional expander 270 may be employed in a longer arm therapeutic compression apparatus 200 such as on a taller individual with a longer arm 500. Any other type of expander may be employed for comfort of the user when the arm therapeutic compression apparatus 200 is secured on the user's arm 500 and the bladder 202 is inflated with a compression profile. Further, the expander 270 may enhance the user's comfort when bending his or her arm 500 at the elbow when the arm therapeutic compression apparatus 200 is secured on the user's arm 500 and the bladder 202 is inflated with a gradient or non-gradient compression profile. Other expanders may be included in the arm therapeutic compression apparatus 200 based on the length of the target limb such as the arm 500.

Referring now to FIGS. 7-11, arm therapeutic compression apparatus 200 includes a compression sleeve 210 connected to a wrap 220. The user connects the compression sleeve 210 to the wrap 220 via a connecting means such as for example inserting at least one button 213 into the opening or aperture 223 on the wrap 220. Once connected the user places the compression sleeve 210 onto the arm 500 by first placing it over the hand 500d then pulling it up over the wrist 500c, elbow 500b and up to the shoulder are 500a. The entire therapeutic compression apparatus is pulled up as the wrap 220 is connected via the connecting means, in this example the buttons 213 and openings or slits 223. Once the user is satisfied with the location of the compression sleeve 210 and connected wrap 220, the user then, with only one hand, closes the wrap 220 via the hook and loop 222, 224, and inflates the bladder 202 so that the therapeutic compression apparatus 200 is not able to shift out of place, thus increasing comfort and reducing fitting issues on the patient. The therapeutic compression apparatus 200 is easier for the patient to apply or place on the arm, notably only using the other hand to do so, as it is one integral apparatus in the embodiment shown in FIGS. 7-11. The therapeutic compression apparatus 200 is placed at a location of the target body part and simultaneously the user can close the wrap 220 via the hook and loop fasteners 222, 224 into a closed position. The secured and closed wrap 220 is then inflated and thereafter remains secure on the arm and has decreased slippage down from the shoulder 500a to the elbow 500b or wrist 500c compared to conventional known compression sleeves or other known wraps or garments.

In another exemplary embodiment, a torso strap (not shown) chest strap (not shown), or a waist strap (not shown) is configured and adapted to improve wearability of therapeutic compression apparatus 200 by locating a portion of bladder 202 above the widest portion of the arm of a patient and provides stability of therapeutic compression apparatus 200 by preventing therapeutic compression apparatus 200 from slipping down the arm of a patient towards the elbow, wrist or hand, which would make the therapeutic compression apparatus 200 less effective in providing arm compression. The inflation means if an automatic pump 150 can be secured on the user via the belt system 180 shown in FIGS. 10-11 and also can be stored in a bag (not shown) which can be worn by the user when moving around such as a string bag, backpack, pocketbook, fanny pack, or any other wearable means which is capable of holding the automatic pump 150 with an aperture for hose 160 to exit the bag or wearable means for the hose 160 to connect to the inflation port 240 on the wrap 220.

Referring now to FIG. 12, there is illustrated an exemplary embodiment of a thigh therapeutic compression apparatus 300 according to the subject invention showing compression bladder 302 integrally formed in the thigh therapeutic compression apparatus 300. The compression bladder 302 includes a medial bladder portion 302A and a lateral bladder portion 302B. The medial bladder portion 302A when in use is located along the medial saphenous vein of the individual on the inner thigh and the lateral bladder portion 302b when in use is located along the lateral saphenous vein of the individual on the back thigh. The thigh therapeutic compression apparatus 300 as shown in FIG. 11 is configured and adapted to wrap around a patient's limb such as in this instance a left thigh, though it could be used in other limbs. An embodiment not shown of the thigh therapeutic compression apparatus 300 is configured and adapted to wrap around a patient's limb such as in this instance a right thigh and would be the mirror image of the inventive thigh therapeutic compression apparatus as shown in FIG. 11 so that the medial bladder 302A would be located on the inner thigh area of the right leg and the lateral bladder portion 302B (if included) would be located on the back thigh of the user. The thigh therapeutic compression apparatus 300 is formed out of continuous outer sheet and inner sheet. The thigh therapeutic compression apparatus 300 is a wrap member with a proximal end portion (top as oriented in FIG. 11) and opposed distal end portion (bottom as oriented in FIG. 11) which is configured and adapted to conform around a patient's thigh and provide compression through the inflation of bladder 302. Inner sheet and outer sheet are made out of a nylon laminated polyurethane sheet which are configured and adapted to be RF welded together. However, any other suitable materials which are weldable or otherwise joined while being airtight can be used. Continuous peripheral weld line 318 forms an airtight boundary of integrally formed bladder 302. In this exemplary embodiment, bladder 302 is a single continuous bladder throughout however, it is envisioned that the thigh therapeutic compression apparatus 300 could have an independent bladder either separately inflatable or inflatable through a one-way valve or other desired inflation/deflation configuration.

In another embodiment not shown, the gap 303 in the lateral bladder 302B (which connects the flow of fluid between 302A and 302B) may be closed off and either the lateral bladder 302B inflated at manufacture to a set pressure level or not inflated and remain in an uninflated state when in use. Also not shown, in another embodiment, the lateral bladder 302B may be closed via a closing means (not shown) such as snap, hook, zipper, or other closing means. In such embodiment, if the lateral bladder 302B is closed or sealed, the lateral bladder 302B may also have a separate inflation means 340′ so that the lateral bladder 302B has a different pressure level than the pressure within the medial bladder 302A. In other embodiments not shown, the lateral bladder 302B may be separately inflatable or inflatable through an inflation means 190 or 150 connected to or inserted within the inflation port 340, such as but not limited to a one-way valve or other desired inflation/deflation configuration. The lateral bladder 302B can also be configured and adapted to provide a differing pressure from the medial bladder 302A. The wrap 320 may not include any bladder in another embodiment not shown.

This embodiment of the present invention includes a therapeutic compression apparatus 300 capable of being secured on the thigh of a user. The thigh therapeutic compression apparatus 300 according to the subject invention includes an elastic sleeve or elastic sock 310 connected to a wrap 320 together integrally formed in the therapeutic compression apparatus 300. The therapeutic compression apparatus 300 is configured and adapted to wrap around a patient's limb such as in this instance as shown in FIG. 12 a thigh from the hip to above the knee 610, though it could be used in other limbs. The therapeutic compression apparatus 300 has an elastic sleeve 310 connected to a wrap member 320 with a proximal end portion located nearer the shoulder hip or waist (top portion as oriented in FIG. 12) and opposed distal end portion nearer the knee (bottom portion as oriented in FIG. 12) which in this embodiment is configured and adapted to conform around a patient's thigh and provide compression through the inflation of bladder 302 (302A, 302B). The elastic sleeve 310 could include additional compression portions (not shown) within similar to the compression portion 112 on the elastic sock or sleeve 110 of leg apparatus 100.

The elastic sleeve 310 is elastic and can be compression or non-compression. The elastic sleeve 310 may be comprised of materials such as nylon, wool, cotton or any other compatible elastic material. The elastic sleeve 310 may have a feature of compression itself which is then another level of compression coupled with the pressure of the bladder 302 (302A, 302B) once inflated. The elastic sleeve 310 is connected via a connecting means to the wrap 320. The connecting means may be via adhesion, sewing, welding, gluing, snaps, hooks, buttons, Velcro™ or any other known connecting means.

In this embodiment, hook fasteners 322 are provided along the edge of inner and outer sheets in order to ease adjustment and secure therapeutic compression apparatus 300 on a patient's limb such as for example a thigh by connecting to the loop portion 324. It is envisioned that the therapeutic compression apparatus 300 can also be secured to a patient's thigh by other means, such as zippered, buttoned, or be cuff shaped by other such suitable means. Further, it is also envisioned that hook 322 and loop closures 324 can be replaced by material such as a hip or waist stabilizer described below and be welded/sewn/attached to bladder 302 for improved comfort.

The thigh therapeutic compression apparatus 300 includes a panel 321 located along the proximal end portion of the thigh therapeutic compression apparatus 300 configured to conform to the groin area of the leg 600. The panel 321 may be comprised of a flexible material such as but not limited to nylon, polyurethane, cotton, or other suitable material, or a material such as Spandex. The panel 321 may be configured to prevent the occurrence of a pinch point and reduce pressure on a patient's groin area when wearing the thigh therapeutic compression apparatus 300.

Referring now to FIGS. 12, by forming bladder 302 (302A and 302B) to be integral within inner sheet and outer sheet, the location and desired preconfigured compression gradient profile can be obtained cost-effectively. A number of different embodiments of bladder configurations can be used in the thigh therapeutic compression apparatus 300 of the subject invention such as those configurations described above. The therapeutic compression apparatus 300 in this embodiment has bladder 302A, 302B with a plurality of spot welds 314 therein. Spot welds 314 are strategically placed within bladder 302 (302A and 302B) in a predetermined pattern based on the desired gradient profile relative to the compression needed at the patient's treatment site. Spot welds 314 enable bladder 302 (302A and 302B) to define the gradient profile when inflated through inflation port 340. The geometric placement of spot welds 314 within bladder 302 allows increased inflation of certain portions of bladder 302 (302A and 302B), and can create one or more fluid chambers within bladder 302. In this embodiment fluid chambers the bladder 302 is comprised of two bladders with air flowing between each bladder, for example the medial bladder 302A located in use along the inner portion of the patient's thigh and the lateral bladder 302B located in use along the patient's back and/or outer thigh. In this exemplary embodiment, bladder 302 is a single continuous bladder comprised of two portions of a bladder such as the medial bladder 302A and lateral bladder 302B. This configuration is particularly useful when compression is needed to improve fluid movement (e.g., blood, lymph, etc.) within the body. Further, linear weld lines 316 allow for better compression along the inner though and outer thigh of a patient's thigh by increasing tension applied to the medial portion of the thigh of a patient and thus further forming the predetermined gradient compression profile. This increased tension can generate a more effective thigh compression in order to increase venous flow. Linear weld lines 316 located horizontally along the medial portion of the thigh create a ribbed portion, which keeps the inflated profile of therapeutic compression apparatus 300 compact which can further increase ambulation and reduce interference with a patient's clothes. While not shown, the linear weld lines 316 could also be located vertically along the medial bladder portion 302A and/or the lateral bladder portion 302B, such as the vertical linear weld lines 316. Other combinations of linear weld lines 316 and spot welds 314 may be used to create a predetermined gradient compression profile for the bladders 302, 302A, 302B, and thus apply gradient compression onto the leg or other limb, body part when in use.

In this embodiment, a further stabilizing means or securing means is comprised of a material that is comfortable to the user, and possibly thin so as to worn either under pants or over pants. The stabilizing mean or securing means is connected to the wrap along the side closest to the lateral bladder portion 302B and from the proximal end to the distal end of the wrap. The stabilizing means or securing means is comprised of cotton, doe skin, leather, rubber, Spandex™ or any other material. The stabilizing means or securing means as shown in FIG. 12 includes a panel 324 and at least two apertures 338A, 338B on the proximal end configured wherein a belt 180 or other securing means can be inserted and then secured around the waist of the individual when in use In this embodiment of the thigh therapeutic compression apparatus 300, the securing means is a panel 324 which includes at least one aperture 338 or two 338A, 338B, created by a linear 339 weld(s) or other sealing means such as a linear stitch, linear adhesion, linear fold, or the like. The apertures 338A, 338B are configured, in this embodiment, to allow a belt system 180 to be inserted within one of the apertures 338A, 338B so as to secure the thigh therapeutic compression apparatus 300 to the waist of a patient and to reduce slippage of the thigh therapeutic compression apparatus 300 down towards the knee.

While only one aperture 338 may be employed (not shown), the configuration including at least two apertures 338A, 338B is suggested so that each user can adjust the placement of the wrap and inventive thigh therapeutic compression apparatus along the user's thigh given the user's height, length of leg, waist, and the like. Other configurations may also be employed having three or more apertures 338. In the embodiment shown in FIG. 12, there are two apertures 338A, 338B so that the patient can adjust the location of the thigh therapeutic compression apparatus 300 on the patient's thigh depending on the height of the patient, the length of the patient's waist (for example, does the patient have a long torso or high or low waist), the length of the patient's thigh (for example, does the patient have long or short legs, including a long or short thigh), the width of the patient's waist (for example, is the patient thinner or thicker at the waist), the anatomy of the patient's hips and buttocks (for example, does the patient have a flat buttock or not), and any other anatomical configuration which may affect how the thigh therapeutic compression apparatus 300 would lie on the patient's thigh when in use and inflated. In use, the patient inserts the belt system 180 into the aperture 338 chosen, either 338A or 338B (or in other embodiments not shown there may be three or more apertures), and loops the belt around the patient's waist, adjusts the length of the belt system 180 via the adjusting means 182, and then cinches or closes the belt such as via a belt buckle 181B and the inserting portion 181A. Again, other securing means may be employed which are not shown such as a waist strap or a hip strap, so as to reduce slippage of the thigh therapeutic compression apparatus 300 downward towards the patient's knee when in use.

In this embodiment, the user connects the compression sleeve 310 to the wrap 320 via a connecting means such as for example inserting at least one button 313 into the opening or aperture 323 on the wrap 320. Once connected the user places the compression sleeve 310 onto the thigh by first placing it over the foot then pulling it up over the ankle, calf, knee 610 and up to the thigh area. The entire therapeutic compression apparatus is pulled up as the wrap 320 is connected via the connecting means, in this example the buttons 313 and openings or slits 323. Once the user is satisfied with the location of the compression sleeve 310 and connected wrap 320, the user then, with only one hand or using both hands, closes the wrap 320 via the hook and loop 322, 324, and inflates the bladder 302 so that the therapeutic compression apparatus 300 is not able to shift out of place, thus increasing comfort and reducing fitting issues on the patient. Further, in order to increase the ease of ambulation by a patient, in this exemplary embodiment, the proximal portion (upper edge located closer to the hip and pelvis) of the thigh therapeutic compression apparatus 300 includes a securing means, so as to stabilize and secure the thigh therapeutic compression apparatus 300 to the thigh of the user and reduce slippage of the thigh therapeutic compression apparatus 300 down towards the knee. The belt system 180 is inserted into one of the apertures 338, 338A, 338B and the belt is placed on the user's waist. The user adjusts the belt by tightening or loosening the adjusters 182 and when satisfied, inserts the male portion 181A of the belt lock into the female portion 181B of the belt lock 181. If an automatic pump 150 is used and placed on the belt 180, an optional pump cover 170 may be placed over the pump 150.

The therapeutic compression apparatus 300 is easier for the patient to apply or place on the thigh, notably only using only one hand or both hands, as it is one integral apparatus in the embodiment comprised of the elastic sock or sleeve 310 and the wrap 320. The therapeutic compression apparatus 300 remains secure on the thigh and has decreased slippage down from the hip to the knee area 610 compared to conventional known compression sleeves or other known wraps, apparatus or garments.

The inflation means or mechanism for each of the various embodiments of the present invention may include a hand pump, electric pump, battery-operated pump, remote controlled pump, airpump, gas pump, or any other known inflation means. A number or variety of inflation means can be employed such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a certain pressure within the bladder so to provide compression in use by the patient. Further, the inflation means could include a means to monitor or regulate the inflation. The inflation means could include programming such that the bladder 102, 202, 302, 302A, 302B is inflated and deflated to a set pressure at intervals or at set times throughout the day or night when the compression apparatus is in use worn on the patient. For instance, by way of example only, the inflation means could be set to 40 mm-Hg at 9 am and then set to deflate to 20 mm-Hg at 11 am and then set to inflate to 30 mm-Hg at 12 pm and so on throughout the day and night for each patient individually. In another embodiment of the present invention not shown there may be two separate bladders 102A and 102B with two separate inflation ports that are each connected to either the same or different inflation means, and the pressure levels of the bladder 102A and the bladder 102B could be the same or have different pressure levels. For instance, by way of example only, the inflation means could be set to 40 mm-Hg for the bladder 102A and set to 20 mm-Hg for the bladder 102B, or each could vary and the inflation means be set to inflate, deflate, inflate, etc. throughout the day as described above.

It is envisioned, that the inventive therapeutic compression apparatus 100, 200, 300 could be used in conjunction with a lower leg compression apparatus (such as those described in U.S. Pat. Nos. 9,033,906 and 7,967,766 and 7,559,908 and 7,276,037 and U.S. Ser. No. 13/444,600), and one inflation means could be connected to each of the two compression apparatus. The inflation means could include programming such that the bladders of both the therapeutic compression apparatus and the bladder or bladders of the lower leg compression apparatus are each (or together at the same time) inflated and deflated to a set pressure at intervals or at set times throughout the day or night when the compression apparatus is in use worn on the patient. In another embodiment of the present invention, for instance, by way of example only, the inflation means could be set to 40 mm-Hg for the thigh bladder and set to 20 mm-Hg for the lower leg bladder (or calf bladder or foot bladder, etc.), or each could vary and the inflation means be set to inflate, deflate, inflate, etc. throughout the day as described above. Depending on the treatment plan for the patient and depending on the specific patient, each of the bladders could be set to the same or different pressure levels though out the day and night.

As an example, treatment options would include intermittent pneumatic compression (HPIPC) 60 minutes twice daily for 16 weeks. In this example, the inflation means would have a pressure level of 120 mm-HG and the bilateral pressures of 120 mm Hg in a cycle time to provide sequential compression for 4 seconds (+/−0.5 seconds) followed by a 16-second rest period (+/−3.0 seconds), resulting in a 20-second cycle or 3 cycles per minute. This treatment may help reduce vascular issues such as lymphedema, DVT, CVI and the like.

In another example, treatment options could include reducing DVT through applying pressure pre-operation, for instance for knee surgery, TKR, KRA, hip surgery, THR, HRA, shoulder surgery, rotator cuff surgery, SLAP surgery, breast cancer surgery, dialysis, or the like, to the target limb for a period of days or weeks or months prior to the surgical date. Prior to surgery the patient would apply compression via the therapeutic compression apparatus 100, 200, 300 and inflation means of the inventive system to reduce swelling of the target limb. During the actual operation and immediately after surgery while still in the hospital setting, the patient would use the same or a different therapeutic compression apparatus 100, 200, 300 connected to an inflation means within the hospital such as an intermittent compression pump accessible through the wall of the patient room or wheeled into the room, which may be considered mechanical DVT prophylaxis. Once discharged from the hospital setting, the patient can retain the same therapeutic compression apparatus 100, 200, 300 and use it at home by applying pressure via the inflation means of an electrical pump 150 configured to apply both constant static pressure and intermittent varying pressure based on the treatment plan. The method of use may reduce or prevent lymphedema, DVT, CVI and other vascular issues. The therapeutic compression apparatus has a universal inflation port configured so it can connect to a hand pump (410) and electric pump (150) and a hospital pump (not shown) and many other pumps. This method if use of the inventive system may reduce swelling pre-surgery and post-surgery and also reduce lymphedema, CVI, DVT and other post-surgical complications. As stated above, the patient is more mobile and ambulatory using the inventive system including the inflation means 150 attached to the retaining means 180 belt. This system is also less cumbersome than current post-operative compression systems which weigh many pounds on their own and other products on the market.

In another embodiment method of use of the inventive system the system us used both prior to, during and after a lower body surgery. Particularly with joint replacement (knee, shoulder and hip) surgery and breast cancer surgery, there is an increased risk of DVT along with the pre and post-op issues related to leg swelling and arm swelling. Due to the universal nature of the inflation port 140, 240, 340 (or valve) on the therapeutic compression apparatus 100, 200, 300, it can be coupled with a hand pump, a battery-operated pump, or the existing electrical intermittent pneumatic compression DVT systems that exist on the market. However, the sleeves to these existing DVT systems are not worn to reduce swelling, cannot apply compression in of a static nature, and cannot be used for mechanical DVT prophylaxis without the IPC DVT control unit. Thus, another method of use to improve the outcomes includes the steps of (a) at least 3 days pre-surgery, the patient is given the therapeutic compression apparatus 100, 200, 300 including the elastic compression stocking or sleeve 110, 210, 310 to reduce limb volume and leg swelling, which can be achieved with either static or intermittent compression pressure levels, then (b) during surgery and immediately post-operative the therapeutic compression apparatus 100, 200, 300 is connected to the IPC DVT control unit in the hospital or clinic or outpatient office setting, and the IPC cycles intermittently to provide standard DVT mechanical prophylaxis, and thereafter (c) as the patient is discharged, the patient is given the therapeutic compression apparatus 100, 200, 300 to bring home along with either or both the hand compression pump or battery operated (or electrical powered) inflation means, such as an automatic pump 150, to manage the risk of DVT along with preventing further swelling.

Further, the therapeutic compression apparatus may be deflated by a valve cap or in another embodiment has a button or a switch to deflate the bladder 102, 202, 302, and thus release the pressure. In another embodiment (not shown), the switch may have a plurality of integrated umbrella valves so that one umbrella valve is set and closed to maintain the pressure within the bladder 102, 202, 302 while a second umbrella valve would release a certain amount of air or fluid within the bladder 102, 202, 302, so as to release the pressure such as while the patient is walking (pressure increases on the leg with each step) or flying (pressure increases based on altitude), and a thirds umbrella valve which would release all the air or fluid in the bladder 102, 202, 302 and thus release all pressure and deflate the therapeutic compression apparatus 100, 200, 300. For instance by way of example only, the first umbrella valve is set in a closed position so that when activated this umbrella valve maintains the amount of air or fluid in the bladder 102, 202, 302 and thus maintains the set pressure, say for instance at 45 mm-Hg, the second umbrella valve is set to release the air or fluid within the bladder 102, 202, 302 if the pressure within exceeds 45 mm-Hg and bring the pressure down to 45 mm-Hg (such as when in high altitude or other increases in pressure) and then maintain the pressure at 45 mm-Hg, and a third umbrella valve is set to open and release all the air or fluid within the bladder 102 and thus release all pressure when activated by the patient so as to deflate the bladder 102, 202, 302 and the therapeutic compression apparatus 100, 200, 300. By way of another example, the dial may include graphics such as (A) “Walk” wherein the set pressure amount is maintained while the patient walks and the pressure spikes and returns over and over in time as the umbrella valve remains in the closed position, (B) then a graphic of “Air” wherein the set pressure amount will be maintained by this umbrella vale occasionally releasing pressure as the pressure increases over the set amount or value so that the umbrella valve is activated to release air or fluid within the bladder 102, 202, 302, and release the pressure yet then close and stay closed to maintain the set pressure amount, and (C) “Release” or “Deflate” wherein the pressure will be released and the air or fluid within the bladder 102, 202, 302, released to deflate and this umbrella valve is always in the open position. In this embodiment (not shown) there are three umbrella valves with one set to always open the bladder 102, 202, 302, to release pressure completely, one set to always close to maintain air or fluid in the bladder 102, 202, 302, to maintain pressure, and a third set to open or release at a predetermined or set pressure point. In all of the embodiments referring to umbrella valve the umbrella valve may also be a switch (manual or otherwise) or a digital switch or any other known means to open, close or partial release air or fluid within a bladder and thereby maintain, change or release pressure therein.

Each of the embodiments of the therapeutic compression apparatus 100, 200, 300 may further (now shown) include an absorbent foam, sponge or dressing to be used alone or with a suction conduit coupled to a suction apparatus. Other embodiments of the compression mechanism are possible such as a combination of a sealed fluid-filled bladder, an absorbent foam, sponge or dressing, and a suction conduit for use with venous ulcers or other open wounds. The absorbent foam, sponge or dressing (not shown and hereinafter referred to as “the sponge”) may have the ability to absorb exudate from the wound or ulcer. In addition, the sponge preferably has an open-cell structure which aids in wicking the exudate from the wound or ulcer. The sponge may be lightly affixed to the patient's skin by an adhesive film such that the sponge may be peeled off and replaced. Some embodiments may include a suction conduit for example provided in between the therapeutic compression apparatus 100, 200, 300 and the sponge. More particularly, the suction conduit may be oriented downwardly towards the sponge so that when a source of negative pressure is applied to the suction conduit, that negative pressure is applied to the wound or ulcer via the sponge. As a result, exudate can be wicked and suctioned away from the wound or ulcer via the sponge and into the conduit (such as a tube and other devices). In one embodiment, the conduit may be easily detached from the therapeutic compression apparatus 100, 200, 300 so that the patient can walk or be transported away from the source of suction while pressure is still being applied to the wound orulcer.

The inventive therapeutic compression apparatus 100, 200, 300 may be included in a kit having various wound dressings and/or bandages. The wound dressings and/or bandages may be disposed of on a more frequent basis and the inventive therapeutic compression apparatus is applied in conjunction or combination with the wound dressings and/or bandages. In one embodiment the therapeutic compression apparatus is used over or on top of the wound dressing applied to the skin.

Another embodiment of the present invention includes a method of applying a measured compression amount with feedback. In this embodiment (not shown), Compression Bladder A is inflated by Inflation Source C—the nature of Compression Bladder A is such that the amount of compression is determined by the amount of inflation medium (typically air) pumped into A from C. In this design, Inflation Source C is also coupled with Bladder B, which has a fixed volume of air. When Compression Bladder A inflates, it will squeeze Bladder B as it compresses Compressed Item F. Inflation Source C is able to read the line pressure from the Coupling Line E to determine the interface pressure from Bladder B—in this design, Inflation Source C can be calibrated to provide only the amount of inflation medium necessary into Compression Bladder A as determined by matching the desired interface pressure from Bladder B. Other configurations may be employed so that feedback may be obtained from the inflation means and compression apparatus.

Another embedment of the present invention includes a Sequential Gradient Compression with Single Chamber. In such embodiment (not shown), the compression apparatus includes an inflation bladder to apply not only gradient compression but sequential (filling up either the bladder 102A first and then filling up the bladder 102B second, or the opposite) compression. In this embodiment (not shown), Inflation Device A is coupled to the device in two places (Intake Port C and Exhaust Port D). By inflating and providing air that goes directly into Channel B, the First Bladder Portion E (either the bladder 102A or 102B as chosen by the patient) inflates first, before the Second Bladder F (the opposite bladder of First Bladder Portion E, so either the bladder 102B or 102A). Air Then exits out of Exhaust Port D. In this design, the inflation can be intermittently provided for gradient and sequential compression or inflation can be held at a constant level to provide just the gradient profile.

Another embodiment of the present invention includes an electric or other automated inflation means such that the bladder is inflated to a set volume or by reading the back pressure of which is being filled in. A pressure cycling function may be included. Further, an embodiment may have an inflation means such that the inflation maintains in the bladder(s) even after the inflation means is removed. Such inflation means may be integral to the compression apparatus itself or may be removable. Such inflation means may include an integrated circuit and/or wireless capability for tracking of usage, pressure, compliance by the patient in regard to maintaining certain pressures recommend by a physician or part of such patient's treatment plan, and other health data such as standing pressure and moving or working pressure, pedometer (number of steps), heartbeat, blood pressure and any other possible monitoring of the patient. Depending on the feedback obtained the inflation means may be programmed to increase or decrease the pressure without manual changing by the patient. Further, the inflation means may be configured so that the physician or other treatment professional may increase or decrease the pressure remotely based on the feedback. Other combinations may be included such as manual changing of the dial or inflation means in combination with automated means or electric means or digital means. A motorized pump and digital display may be used. The valve may include digital or electric means to change or modify pressure at a set rate or intervals or based on feedback from the monitoring means. The apparatus may include various sensors and monitors.

Post-operative treatment can also be effective with therapeutic compression apparatus 100, 200, 300 including without limitation knee replacements, hip replacements, shoulder replacements, rotator cuff surgery, breast cancer surgery, SLAP, sclerotherapy, dialysis and other procedures. Other treatments such as lymphedema may employ the therapeutic compression apparatus 100, 200, 300 so as to apply compression and pressure to the selected site on the patient's thigh or lower leg. In use the therapeutic compression apparatus 100, 200, 300 may be placed by the patient, practioner or care-giver on the chosen limb, such as for instance the lower leg, thigh, arm or other target body part or limb. The elastic sock or sleeve 110, 210, 310 is connected to the wrap 120, 220, 320 and then placed by the user on the foot and pulled towards the knee or placed on the hand and pulled towards the shoulder until reaching the satisfactory location on the body part. The therapeutic compression apparatus is thus located on the target body area and secured due to the compression of the elastic sock or sleeve 110, 210, 310 allowing the user to simultaneously now close the wrap 120, 220, 320 around the target body part by moving in an upward or downward position the patient, practitioner or care-giver fastens or secures the fastening tabs such as 122, 124 up or down to the knee and ankle or wrist to shoulder. If there are additional optional straps located on the proximal end of the apparatus the first strap should be closed or secured in a tight fashion so that the therapeutic compression apparatus fits snugly but not too tight and the second strap should be closed or secured in a tight fashion so that the therapeutic compression apparatus fits snugly but not too tight. The patient, practioner or care-giver then removes the valve cap 144, 244, 344 from the inflation port 140, 240, 340 on the therapeutic compression apparatus 100, 200, 300. The patient, practioner or care-giver then selects a pressure amount of value on the dial of the inflation means depending on the treatment and whether the patient will be walking, sitting, lying down or traveling in a vehicle, train or airplane. Once the pressure amount or value is chosen on the dial (such as a given pressure amount such as “35” mm-Hg or a text such as “Walk” or “Air” or “Travel” or “Low” or “Medium” or “High”), the corresponding umbrella valve or switch is activated such that the pressure is thereafter maintained (closed position) or modified so as to maintain the pressure as it changes with the activity or altitude when in use). The patient, practioner or care-giver then inserts an end of the inflation means into the valve on the therapeutic compression apparatus and the air or fluid is increased to inflate the bladder (102, 202, 302) and thus achieve a desired pressure amount or valve. Again this inflation means may be a hand pump, electric pump, battery-operated pump, remote controlled pump, air pump, gas pump, or any other known inflation means. At any point in use the patient, practioner or care-giver can deflate the bladder 102, 202, 302 by either inserting the valve cap 144, 244, 344 so it depresses the valve spring and thus release the air or fluid in the bladder and decrease the pressure, or the patient, practioner or care-giver can reinsert the inflation means and select the “Deflate” or “Release” and the corresponding umbrella valve will be in the open position so as to release the air or fluid in the bladder 102, 202, 302 and decrease the pressure and/or compression until a deflated state is achieved for the bladder 102, 202, 302 and the therapeutic compression apparatus 100, 200, 300. The therapeutic compression apparatus 100, 200, 300 can be reinflated and deflated over and over again when in use.

Another embodiment (not shown) may be a full leg compression apparatus including a thigh bladder, a calf bladder, an optional foot bladder, and a knee opening (or an optional knee pad) and an optional heel opening and foot strap. In this configuration, the thigh bladder may extend below the knee joint level and apply compression around the patella of the knee area. It may also have suspender hooks or slots (not shown) that allow for suspenders to be mated thereto in order to support the apparatus by a waist band when in use. The suspenders limit downward travel of the apparatus when in use such that it maintains its desired position.

The present invention has been illustrated and described with respect to specific embodiments thereof, which embodiments are exemplary and illustrative of the principles of the invention and are not intended to be exclusive or otherwise limiting embodiments. For instance, while in the foregoing embodiments the therapeutic compression apparatus are described as having inflatable bladders, the therapeutic compression apparatus may additionally include integrally formed or attached (e.g., by adhesive, radio-frequency welding, etc.) compression members that are not configured for inflation and/or deflation. For instance, additional compression members may be implemented using any of a variety of preformed and/or prefilled cushioning materials such as foam cushions and/or air, gel, or other fluid filled non-inflatable cushions, provided such compression members generate sufficient compression in combination with integral compression bladders. Further, while particular shapes, sizes, and materials have been described for purposes of illustration, it will be recognized that any of a variety of shape or size can be used, and the materials described are not exclusive but merely illustrative. Also, as noted above, while the bladder shown is inflated with air, it will be appreciated that any other fluid or medium such as liquid or gel can be used. Moreover, as also noted, it will be understood that bladders may be configured to have multiple pneumatically independent and/or pneumatically coupled bladder sections, and may also be configured to have various contours or lobulations.

The therapeutic compression apparatus described herein can be used for any suitable condition treatable by compression therapy and the like. For example, therapeutic compression apparatus in accordance with the present invention can be used for compression of the venous system for the treatment of venous ulcers, CVI, DVT, for the treatment of lymphedema (where it is circulation of fluids in the lymph system rather than in the venous system that is promoted), and the like. It can be used to fulfill a long felt need while a patient is undergoing dialysis or after to reduce or prevent swelling of the arm or other body part as well as lymphedema, CVI or DVT. It can be used to fulfill a long felt need for a compression wrap, garment, apparatus or system to increase warm up before exercise, to remove lactic acid buildup during and after exercise and to reduce recovery time for the user.

The therapeutic compression apparatus of the instant invention described herein solves many problems with the prior art and in the industry and treatment of patients. The therapeutic compression apparatus may be applied on the patient's body part by the patient without the need or requirement of a skilled care-giver as required by current devices and apparatus. The ease of use by the patient in applying the apparatus to the limb is an advantage over the known prior art. The inventive therapeutic compression apparatus 100 is capable of maintaining sufficient effective pressure without overpressure complications, maintaining compression and the like. The user can simultaneously locate the inventive therapeutic compression apparatus on the target body part while at the same time closing the wrap such as connecting fasteners or VELCRO™ straps and other connectors without the wrap moving off the target body part.

The therapeutic compression apparatus of the instant invention includes a universal inflation port which is configured to be capable of connecting to more than one source of compression or inflation means such that the patient could vary treatment through varying the inflation source and inflation means for the treatment apparatus or device. For instance, a patient using the therapeutic compression apparatus of the instant invention can alternate between a manual or mechanical or electrical inflation means or source of inflation and pressure. Further, the patient can alternate between static or intermittent inflation and pressure when using the inventive therapeutic compression apparatus.

The therapeutic compression apparatus of the instant invention also fulfills a long-felt need to reduce the problem is leakage of set compression within the treatment apparatus and devices, bandages, stockings and hosiery and instead promotes a more effective treatment for CVI, DVT and/or lymphedema and other treatments. The therapeutic compression apparatus of the instant invention also fulfills a long-felt need and reduces the problem of slippage downward towards the knee or ankle of the patient when the apparatus is in use on the thigh or lower leg, especially when the patient is walking or moving about.

While in recent years various products have been created that include tight compression stockings or sleeves, these products have many problems as discussed above with being too tight to maintain location and non-slippage and then stretching out over time. Further, products which allow the user to apply a aster such as VELCRO™ to create compression with or without an inflation means, these products have problems in ease of location on the target body part while being closed at the same time by the user and then continued problems with slippage down to the knee, ankle or wrist. The inventive therapeutic compression apparatus and system do not have such problems and fulfill the long felt need for a compression apparatus which can be simultaneously placed on a location at a target body part and closed at the same time by the user himself or herself and when moving the user will see reduced or no slippage down to the knee, ankle or wrist. The inventive therapeutic compression apparatus 100, 200, 300 has the benefit of an elastic sock or sleeve without the problem of too high elasticity and the added benefit of compression (either from the hook and loop fasteners alone or with inflation of the bladder). These and other needs are met by the inventive therapeutic compression apparatus and system 100, 200, 300.

The inventive therapeutic compression apparatus 100, 200, 300 combined with the belt system 180 and other additions are a system and can be combined with other kits and products. The inventive therapeutic compression apparatus fulfill the need of a self-application by a user of a compression wrap, garment or apparatus even of the user has reduced strength or mobility in his or her hands, arms, legs or back. Once applied the inventive therapeutic compression apparatus and system 100, 200, 300 maintains stability securely on the target body part while maintaining sufficient compression levels on the target body part. The inventive therapeutic compression apparatus and system 100, 200, 300 may be used pre-surgery or post-surgery to reduce swelling and lymphedema as well as during dialysis as well as pre-exercise, during and post-exercise. Other uses of the inventive therapeutic compression apparatus and system 100, 200, 300 may be employed including without limitation treating or reducing CVI and/or DVT.

While the subject invention of the present disclosure has been described with respect to preferred and exemplary embodiments, those skilled in the art will readily appreciate that various changes and/or modifications can be made to the invention without departing from the spirit or scope of the invention as, described herein. There have been described and illustrated herein several embodiments of an intermittent pressure apparatus and a method of installing and operating same. While particular embodiments of the invention have been described, it is not intended that the invention be limited thereto, as it is intended that the invention be as broad in scope as the art will allow and that the specification be read likewise. Thus, while particular shapes and sizes of inflatable bladders and straps have been disclosed, it will be appreciated that other shapes, sizes, and attachment means may be used as well. It will also be understood that while VELCRO™ and adhesive means have been disclosed for helping to secure the bladders to the leg and foot, other types of attachments such as hooks, snaps, or wraps may be used. In addition, it will be appreciated that while the fluid conduit may be detachably connected to the bladders using mating threaded portions or bayonet locks, other means of attachment known in the art may be used. It will therefore be appreciated by those skilled in the art that yet other modifications could be made to the provided invention without deviating from its spirit and scope as claimed.

We claim:

1. A therapeutic compression system for applying pressure to at least one limb of the human body, comprising:
at least one therapeutic compression apparatus including at least one elastic sleeve and at least one wrap configured to be worn on a limb of a user including at least one bladder connected to a universal inflation port configured to be capable of connection to a static or intermittent inflation means, and a connecting means configured to connect the at least one elastic sleeve to the at least one wrap;
a static or intermittent inflation means connected to the at least one bladder via the universal inflation port wherein the inflation means includes a check valve; and
wherein the universal inflation port includes a sealing means configured to include an open position and a closed position wherein the closed position maintains the at least one bladder in an inflated or deflated state and is in the open position when coupled to the inflation means so that the at least one bladder is the same inflation state as the inflation means.

2. The therapeutic compression system of claim 1 wherein the connecting means is selected from the group consisting of buttons, stud buttons, snaps, straps, adhesives, welds, zippers, hooks, buckles, hook and loop, or a combination thereof.

3. The therapeutic compression system of claim 1 further comprising a compression strap configured for the foot and wherein the at least one elastic sleeve is configured to be placed on a lower leg including a closed foot portion of the elastic sleeve.

4. The therapeutic compression system of claim 3 wherein the foot compression strap is configured to be placed around an arch and sole area of the foot over the elastic sleeve.

5. The therapeutic compression system of claim 3 wherein the at least one wrap includes an elastic portion configured to be placed on the ankle portion of a user.

6. The therapeutic compression system of claim 1 wherein the at least one wrap is configured to be placed on an arm of a user and includes an aperture configured to encircle a portion of an elbow portion of a user.

7. The therapeutic compression system of claim 6 wherein the at least one elastic sleeve is configured to be placed on an arm of a user and includes an aperture on a distal end of the elastic sleeve configured for insertion of a thumb of a user.

8. The therapeutic compression system of claim 1 wherein the at least one wrap is configured to be placed on a thigh of a user and includes an elastic portion configured to be placed on the groin area of a user.

9. The therapeutic compression system of claim 1 wherein the inflation means is selected from the group consisting of manual pumps, static pumps, intermittent pumps, electrical inflation pumps, battery inflation pumps, gas powered inflation pumps, static pneumatic compression pumps, intermittent pneumatic pressure pumps, and the combination thereof and the inflation means for the at least one bladder is selected from the group consisting of air, gas, fluid or combinations thereof.

10. The therapeutic compression system of claim 1, wherein the inflation means includes a manual pump configured to provide static inflation.

11. The therapeutic compression system of claim 1, wherein the inflation means includes a powered pump configured to provide static and/or intermittent inflation.

12. The therapeutic compression system of claim 1, wherein the inflation means is connected to a securing means configured to worn on the body of the user.

13. The therapeutic compression system of claim 1, wherein the sealing means is selected from the group consisting of valves, caps, levers, switches, screws, stop taps, stopcocks, or combinations thereof.

14. The therapeutic compression system of claim 1, wherein the inflation port is a self-sealing inflation port configured to prevent deflation of the at least one bladder and includes a check valve.

15. The therapeutic compression system of claim 1 further comprising a pressure sensor operatively connected to the inflation means to protect from over inflation and wherein the check valve is set to open at a predetermined pressure or a user selectable pressure.

16. The therapeutic compression system of claim 1 further comprising a second wrap wherein the first wrap and the second wrap may each be configured to wrap around a limb of the user wherein the limb is selected from the group consisting of the foot, ankle, calf, lower leg, knee, thigh, upper leg, whole leg, waist, torso, chest, arm, shoulder, elbow, wrist, hand, neck or any combinations thereof.

17. The therapeutic compression system of claim 16 wherein the first wrap and second wrap are not connected to each other while connected to the same inflation means and wherein the second wrap has a different inflation pressure than the inflation pressure of the first wrap when both the first wrap and second wrap are inflated from the same inflation means.

18. The therapeutic compression system of claim 1 wherein the at least one wrap includes at least two bladders configured to be either separate or joined.

19. The therapeutic compression system of claim 18 wherein the second bladder has a different inflation pressure than the inflation pressure of the first bladder when both the first wrap and second wrap are inflated from the same inflation means.

20. The therapeutic compression system of claim 1, wherein the at least one bladder is an air bladder configured to have at least one chamber capable of sequential gradient pressure when connected to the inflation means.

21. The therapeutic compression system of claim 1, wherein the at least one bladder is configured to provide gradient compression profile when inflated by the inflation means.

22. The therapeutic compression system of claim 1, wherein the inflation means includes a three-way switch configured to be capable of holding a pressure created by the inflation means within the at least one bladder creating an inflated state of the bladder, capable of releasing a set amount of pressure within the at least one bladder creating a partially deflated state of the bladder, and capable of releasing all the pressure within the at least one bladder so as to create a fully deflated state of the bladder.

23. The therapeutic compression system of claim 1 wherein the inflation means includes areal-time pressure measurement mechanism.

24. A method for applying compression pressure to a portion of a human body, comprising:
activating an at least one therapeutic compression apparatus by connecting at least one elastic sleeve to at least one wrap by a connecting means;
affixing the at least one elastic sleeve on a body part of a user;
closing the at least one wrap on a body part of a user, wherein the at least one wrap includes (a) at least one air bladder connected to an inflation means and (b) an universal inflation port having a sealing means configured to include an open position and a closed position wherein the closed position maintains the at least one bladder in an inflated or deflated state and is in the open position when coupled to the inflation means so that the at least one bladder is the same inflation state as the inflation means, and further including an elbow connector, a check valve and a valve cap wherein the valve cap is capable of releasing a pressure created by the inflation means within the at least one air bladder;
securing the inflation means to the same or different body part of a user; inflating the at least one air bladder;
moving such that the at least one wrap is secure and stable on the body part; removing the inflation means from the universal inflation port;
activating the sealing means on the universal inflation port; and
deflating the inflated at least one bladder by opening the sealing means and releasing a pressure within the at least one bladder.

25. A therapeutic compression system comprising:
at least one elastic sleeve configured to be located on an arm, hip, leg, groin, knee, or foot area of a user;
at least one wrap having a proximal end configured to be located along a shoulder, hip, groin or knee area of a user and a distal end of the primary wrap configured to be located at a wrist, knee or ankle of a user, the primary wrap including at least one bladder;
a secondary wrap having a proximal end and a distal end wherein the proximal end of the secondary wrap is connected to the distal end of the primary wrap;
an optional inflation means connected to the bladder via a universal inflation port on the bladder wherein the inflation port includes a check valve and is configured to be capable of connection to a manual or powered pump; and
an assembly comprising: a) a pressure mechanism having a flexible member for attachment around a limb and an air chamber which assumes a first depressurized state and a second pressurized state, said air chamber having a length and a width, said width being less than half the width of the flexible member; b) a pre-filled air bladder having a length and a width smaller than the width of the air chamber; c) an absorbent foam, sponge or dressing coupled to the pre-filled air bladder; and d) a suction conduit in fluid communication with the absorbent foam, sponge or dressing and adapted for coupling to a source of negative pressure.
